(19) United States
(12) Patent Application Publication
(10) Pub. No.: US 2010/0179105 A1
(43) Pub. Date: Jul. 15, 2010

(54) KERATIN MATERIALS MAKE-UP KIT COMPRISING REACTIVE SILICONE COMPOUNDS AND A COMPATIBLE OIL

(76) Inventors: Xavier Blin, Paris (FR); Alexandra Schvent, Saint-Mande (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/520,083

(22) PCT Filed: Dec. 19, 2007
(86) PCT No.: PCT/EP2007/064272

§ 371 Date: Mar. 5, 2010

Related U.S. Application Data
(60) Provisional application No. 60/878,077, filed on Jan. 3, 2007.

(30) Foreign Application Priority Data
Dec. 20, 2006 (FR) 06/55698

(51) Int. Cl. A61K 8/92

(52) U.S. Cl. 514/63

(57) ABSTRACT

The invention provides a make-up kit comprising at least two compositions which are packaged separately, —the kit comprising at least one compound X, at least one compound Y and at least one catalyst or one peroxide, at least one of the compounds, X or Y, being a silicone compound, —with the proviso that the compounds X and Y—and the catalyst are not simultaneously in one of said compositions, —said compounds X and Y being able to react together by a hydrosilylation reaction or by a condensation reaction, when they are brought into contact with one another, —one at least of the first and second compositions comprising at least one compatible oil, —said compounds X and Y and said compatible oil, and their respective amounts, being such that the transfer index of a deposit comprising the two compositions is less than or equal to 40 out of 100. Finally, the invention relates to a method of making-up keratin materials that involves applying the compounds X and Y and the compatible oil.

The invention relates to a kit comprising two separately packaged cosmetic compositions intended for application to the keratin materials, said kit comprising a compound X or a compound Y, which are reacted together in situ or just before application to the keratin materials. One at least of the compounds X and Y is a silicone compound, and one at least of the two compositions further comprises at least one, preferably non-volatile, compatible oil.

The invention also relates to a method which consists in applying the compounds X and Y and the compatible oil to the keratin materials.

The compositions may be applied in particular to the hair, skin, lips, eyelashes, eyebrows or nails.

The compositions according to the invention may be compositions for making-up or caring for keratin materials.

In particular, each composition may be a make-up product such as a loose or compact powder, a foundation, a blusher, an eyeshadow, a concealer, a rouge, a lipstick, a lip balm, a lipgloss, a lip pencil, an eye pencil, a mascara, an eyeliner or else a body make-up or skin colouring product.

In the case of a care product, each composition may be a face and body skincare, lipcare or lashcare product, more particularly a sun product.

Make-up compositions, when they are applied to the skin, often have the drawback of transferring, in other words of depositing at least partly, leaving traces, on certain substrates with which they may be brought into contact, and more particularly a glass, cup, cigarette, article of clothing, or the skin. A consequence of this is a poor persistence on the part of the applied film, necessitating regular reapplication of the foundation or lipstick.

There is therefore a desire for skin and lip make-up compositions referred to as “non-transfer” compositions, which have the advantage of forming a deposit which is not deposited at least partly on the substrates with which the compositions are brought into contact (glass, clothing, cigarette, fabrics).

For the purpose of limiting the transfer of cosmetic compositions it is known to employ volatile oils. These volatile oils, when present in a large amount, make the make-up product, more particularly lipstick, uncomfortable for the user: the make-up deposit imparts a sensation of drying and pulling.

The aim of the present invention is to provide a new formulation route for make-up compositions that allows a film deposited on the keratin materials to be obtained that has good non-transfer properties, good properties of staying power over time, in particular with respect to water and to rubbing, and which features enhanced comfort.

The inventors have found that it is possible to obtain such properties by using a system comprising silicone compounds which polymerize in situ and particular oils.

Accordingly, in a first aspect, the present invention provides a make-up kit comprising at least two compositions which are packaged separately,
the kit comprising at least one compound X, at least one compound Y and optionally at least one catalyst or one peroxide, at least one of the compounds, X or Y, being a silicone compound,
with the proviso that the compounds X and Y—and the catalyst, when present, or the peroxide, when present—are not simultaneously in one of said compositions,
said compounds X and Y being able to react together by a hydrosilylation reaction, or by a condensation reaction or by a crosslinking reaction in the presence of a peroxide, when they are brought into contact with one another,
one at least of the first and second compositions comprising at least one compatible oil,
said compounds X and Y and said compatible oil, and their respective amounts, being such that the transfer index of a deposit comprising the two compositions is less than or equal to 40 out of 100.

In particular the invention provides more particularly a make-up kit comprising at least two compositions which are packaged separately,
the kit comprising at least one compound X, at least one compound Y and at least one catalyst, at least one of the compounds, X or Y, being a silicone compound,
with the proviso that the compounds X and Y—and the catalyst, are not simultaneously in one of said compositions,
said compounds X and Y reacting together by a hydrosilylation reaction when they are brought into contact with one another in the presence of the catalyst,
one at least of the first and second compositions comprising at least one compatible oil,
said compounds X and Y and said compatible oil, and their respective amounts, being such that the transfer index of a deposit comprising the two compositions is less than or equal to 40 out of 100.

The invention likewise provides a make-up kit comprising at least two compositions which are packaged separately,
the kit comprising at least one compound X, at least one compound Y and optionally at least one catalyst, at least one of the compounds, X or Y, being a silicone compound,
with the proviso that the compounds X and Y are not simultaneously in one of said compositions,
said compounds X and Y reacting together by a condensation reaction when they are brought into contact with one another,
one at least of the first and second compositions comprising at least one compatible oil,
said compounds X and Y and said compatible oil, and their respective amounts, being such that the transfer index of a deposit comprising the two compositions is less than or equal to 40 out of 100.

A silicone compound is a compound comprising at least one or even at least two organosiloxane units. In one particular embodiment the compounds X and Y are silicone compounds.

The compounds X and Y are able to react together on keratin materials or on a substrate in such a way as to form a comfortable non-transfer film on the keratin materials in the presence of the compatible oil.

In one embodiment the present invention provides a cosmetic kit comprising at least two separately packaged compositions,
the kit comprising at least one silicone compound X, at least one silicone compound Y and optionally at least one catalyst,
with the proviso that the compounds X and Y—and the catalyst when present—are not simultaneously in one of said compositions,
said compounds X and Y being able to react together by hydrosilylation reaction when they are contacted with one another,
one at least of the first and second compositions comprising at least one compatible oil,
said compounds X and Y and said compatible oil, and their respective amounts, being such that the transfer index of a deposit comprising the two compositions is less than or equal to 40 out of 100.

Each composition may be packaged separately in a single pack, as for example in a twin-compartment pen, the base composition being delivered by one end of the pen and the top composition being delivered by the other end of the pen, with each end being closed, more particularly closed tightly, by a cap. Each composition may also be packaged in a compartment within a single pack, the mixing of the two compositions taking place at the end or ends of the pack at the time of delivery of each composition.

Alternatively, each of the first and second compositions may be packaged in a different pack.

In one embodiment the compounds X and Y may be present in two distinct compositions referred to as first and second compositions. In this embodiment the compounds X and Y are mixed at the time of use, preferably in the presence of a catalyst, via the mixing of the two compositions, and then the mixture is applied to the keratin materials.

The invention further provides for the use of a kit as described above to give a film that exhibits satisfactory gloss properties while being endowed with enhanced non-transfer properties.

Each composition of course comprises a cosmetically acceptable medium, which is a non-toxic medium which can be applied to the keratin materials of human beings and has a pleasant appearance, odour and feel.

The invention likewise provides a cosmetic keratin material coating method consisting

a) in mixing at the time of use
at least two compositions so as to consociate at least one compound X, at least one compound Y, and optionally at least one catalyst or one peroxide, at least one of the compounds X or Y being a silicone compound,
with the proviso that the compounds X and Y—and the catalyst when present or the peroxide when present—are not simultaneously in one of said compositions,
said compounds X and Y reacting together by a hydrosilylation reaction, or by a condensation reaction or by a crosslinking reaction in the presence of a peroxide, when they are brought into contact with one another,
one at least of the first and second compositions containing at least one compatible oil,
said compounds X and Y and said compatible oil, and their respective amounts, being such that the transfer index of a deposit comprising the two compositions is less than or equal to 40 out of 100;
   b) then in applying to said keratin materials at least one layer of said mixture.

In one version the invention provides a cosmetic keratin material coating method consisting
in successively applying to the keratin materials at least two compositions so as to consociate at least one compound X, at least one compound Y, and optionally at least one catalyst or one peroxide, at least one of the compounds X or Y being a silicone compound,
with the proviso that the compounds X and Y—and the catalyst when present or the peroxide when present—are not simultaneously in one of said compositions,
said compounds X and Y reacting together by a hydrosilylation reaction, or by a condensation reaction or by a crosslinking reaction in the presence of a peroxide, when they are brought into contact with one another,
one at least of the first and second compositions comprising at least one compatible oil,
said compounds X and Y and said compatible oil, and their respective amounts, being such that the transfer index of a deposit comprising the two compositions is less than or equal to 40 out of 100.

In another aspect the compounds X and Y and the compatible oil may be present within a single composition, which in that case comprises
at least one compound X and at least one compound Y, one at least of the compounds X and Y being a silicone compound, said compounds X and Y being able to react together by a hydrosilylation reaction, condensation reaction or crosslinking reaction in the presence of a peroxide, and
at least one compatible oil,
said compounds X and Y and said compatible oil, and their respective amounts, being such that the transfer index of a deposit comprising the two compositions is less than or equal to 40 out of 100.

In this embodiment one at least of the compounds X and Y may be present in an encapsulated form.

In one version the two compounds X and Y are both present in separate encapsulated forms.

More particularly the compounds X and/or Y may be present in the form of microcapsules and more particularly of core/shell nanocapsules, the lipophilic core containing the compound X or the compound Y.

### Compatible Oil

A compatible oil is one which is compatible with the compound X and/or compatible with the compound Y, preferably an oil which is compatible with both X and Y.

A compatible oil is an oil which gives an homogeneous mixture with the compound X or, respectively, the compound Y, when it is in a proportion of less than or equal to 50/50 by weight.

The incompatibility of the compound X with the oil or the incompatibility of the compound Y with the oil may take the form of phase separation of the mixture or of separating-out of the oil in the mixture. The compatible nature of an oil with the compound X or the compound Y is evaluated, in accordance for example with the following protocol, at 25° C.

A defined mass of the compound X or of the compound Y is introduced into a beaker and then the oil is added progressively, with magnetic stirring, in an amount such that an oil/compound X or oil/compound Y mixture in a proportion less than or equal to 50/50 by weight is obtained.

The mixture is then poured into a 20 ml flask, for example a Chromacol 20 CV 548-1194 VB flask for fastening, which is then fastened. After 24 hours, the flask is inspected with the naked eye for the presence of two phases and/or the opacity of the mixture, which are demonstrators of incompatibility.

Under these conditions, for example, octyldodecanol (which is a transparent liquid), in a 50/50 by weight mixture with the mixture X′ (which is an iridescent fluid) provided by Dow Corning gives:

MIXTURE X′:

| Ingredient (INCI name) | CAS No. | Amounts (%) | Function |
|---|---|---|---|
| Dimethyl siloxane having | 68083-19-2 | 55-95 | polymer |
| dimethylvinysiloxy | | | |
| terminations | | | |
| Silica silylate | 68909-20-6 | 10-40 | filler |
| 1,3-Diethenyl-1,1,3,3-tetra- | 68478-92-2 | trace | catalyst |
| methyldisiloxane complex | | | |
| Tetramethyldivinyldisiloxane | 2627-95-4 | 0.1-1 | polymer |

Or with the mixture Y′ (which is an iridescent fluid) supplied by Dow Corning:

MIXTURE Y′:

| Ingredient (INCI name) | CAS No. | Amounts (%) | Function |
|---|---|---|---|
| Dimethyl siloxane, dimethyl- | 68083-19-2 | 55-95 | polymer |
| vinylsiloxy | | | |
| Silica silylate | 68909-20-6 | 10-40 | filler |
| Dimethyl methylhydrogeno | 68037-59-2 | 1-10 | polymer |
| siloxane, having trimethyl- | | | |
| siloxy terminations | | | |

a white opaque mixture which separates out from the oil. In the sense of the present invention, therefore, octyldodecanol is incompatible with each of these two silicone compounds. Under these conditions, for example, octyldodecanol (which is a transparent liquid) gives a 50/50 mixture by weight with the commercial product Dow Corning 7-9800 Soft Skin Adhesive Part A (which is a colourless transparent liquid) or, with the commercial product Dow Corning 7-9800 Soft Skin Adhesive Part A (which is a colourless transparent liquid), gives a two-phase translucent mixture. In the sense of the present invention, therefore, octyldodecanol is incompatible with each of these two silicone compounds.

The compatible oil may be a preferably non-volatile ester oil. A non-volatile oil is an oil which remains on the keratin materials at ambient temperature and atmospheric pressure for at least a number of hours, and more particularly has a vapour pressure of less than 10⁻³ mmHg (0.13 Pa).

This ester oil may be selected from the esters of monocarboxylic acids with monoalcohols and polyalcohols.

Advantageously said ester conforms to the following formula (I):

R₁—CO—O—R₂ (I)

where R₁ represents a linear or branched alkyl radical of 1 to 40 carbon atoms, preferably 7 to 19 carbon atoms, optionally comprising one or more ethylenic double bonds, and optionally substituted.

R₂ represents a linear or branched alkyl radical of 1 to 40 carbon atoms, preferably 3 to 30 carbon atoms and more preferably 3 to 20 carbon atoms, optionally comprising one or more ethylenic double bonds, and optionally substituted.

By “optionally substituted” is meant that R₁ and/or R₂ may carry one or more substituents selected, for example, from groups containing one or more heteroatoms selected from O, N and S, such as amino, amine, alkoxy and hydroxyl.

Preferably the total number of carbon atoms of R₁+R₂ is ≧9.

R₁ may represent the residue of a fatty acid, preferably a higher, linear or, preferably, branched fatty acid containing 1 to 40 and more preferably 7 to 19 carbon atoms, and R₂ may represent a linear or, preferably, branched hydrocarbon chain containing 1 to 40, preferably 3 to 30 and more preferably 3 to 20 carbon atoms. Again, preferably, the number of carbon atoms of R₁+R₂≧9.

Examples of the groups R₁ are those derived from fatty acids selected from the group consisting of acetic, propionic, butyric, caproic, caprylic, pelargonic, capric, undecanoic, lauric, myristic, palmitic, stearic, isostearic, arachidic, behenic, oleic, linolenic, linoleic, eleostearic, arachidonic and erucic acids and mixtures thereof.

Examples of esters are, for example, purcellin oil (cetostearyl octanoate), isononyl isononanoate, isopropyl myristate, 2-ethylhexyl palmitate, 2-octyldodecyl stearate, 2-octyldodecyl erucate, isostearyl isostearate and the heptanoates, octanoates, decanoates or ricinoleates of alcohols or of polyalcohols, for example of fatty alcohols.

Advantageously the esters are selected from the compounds of the formula (I) above in which R₁ represents an unsubstituted linear or branched alkyl group optionally containing one or more ethylenic double bonds, of 1 to 40 carbon atoms, preferably of 7 to 19 carbon atoms, and R₂ represents an unsubstituted linear or branched alkyl group optionally containing one or more ethylenic double bonds, of 1 to 40 carbon atoms, preferably of 3 to 30 carbon atoms and more preferably of 3 to 20 carbon atoms.

Preferably R₁ is an unsubstituted branched alkyl group of 4 to 14 carbon atoms, preferably of 8 to 10 carbon atoms, and R₂ is an unsubstituted branched alkyl group of 5 to 15 carbon atoms, preferably of 9 to 11 carbon atoms. Preferably in the formula (I) R₁—CO— and R₂ have the same number of carbon atoms and derive from the same radical, preferably unsubstituted branched alkyl, for example isononyl; in other words, advantageously, the ester oil molecule is symmetric.

The ester oil will be selected preferably from the following compounds:
isononyl isononanoate,
cetostearyl octanoate,
isopropyl myristate,
2-ethylhexyl palmitate,
2-octyldodecyl stearate,
2-octyldodecyl erucate,
isostearyl isostearate.

The compatible oil may be a non-volatile silicone oil.

The non-volatile silicone oils may be:
non-volatile polydimethylsiloxanes (PDMS),
polydimethylsiloxanes comprising alkyl or alkoxy groups pendently and/or at the end of the silicone chain, said groups each having 3 to 40 carbon atoms,
phenyl silicones such as phenyltrimethicones, phenyldimethicones, phenyltrimethylsiloxydiphenylsiloxanes, diphenyldimethicones, diphenylmethyldiphenyltrisiloxanes, 2-phenylethyl trimethylsiloxysilicates and trimethylpentaphenyltrisiloxane;
polyalkylmethylsiloxanes, optionally fluorinated such as the polymethyltrifluoropropyldimethylsiloxanes;
polyalkylmethylsiloxanes substituted by functional groups such as hydroxyl, thiol and/or amine groups;
polysiloxanes modified with fatty acids, fatty alcohols or polyoxyalkylenes,
and mixtures thereof.

The non-volatile silicone oil may be selected from non-volatile phenyl silicone oils.

The non-volatile phenyl silicone oil may be selected from the phenyl silicones of formula (VI) below:

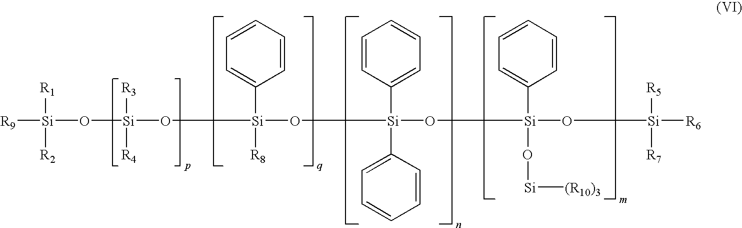

in which
R1 to R10 independently of one another are saturated or unsaturated linear, cyclic or branched C1-C30 hydrocarbon radicals,
m, n, p and q, independently of one another, are integers between 0 and 900, with the proviso that the sum ‘m+n+q’ is other than 0.

Preferably the sum ‘m+n+q’ is between 1 and 100.

Preferably the sum ‘m+n+p+q’ is between 1 and 900, more preferably still between 1 and 800.

Preferably q is 0.

Preferably the non-volatile phenyl silicone oil is selected from the phenyl silicones of formula (VII) below:

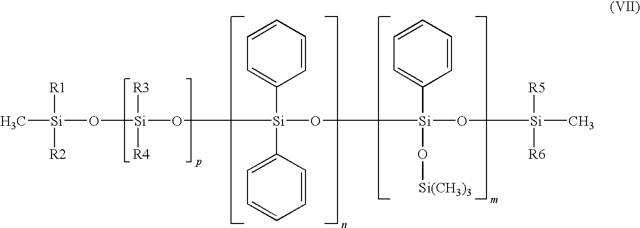

in which:
R1 to R6 independently of one another are saturated or unsaturated linear, cyclic or branched C1-C30 hydrocarbon radicals,
m, n and p, independently of one another, are integers between 0 and 100, with the proviso that the sum ‘n+m’ is between 1 and 100.

Preferably R1 to R6 independently of one another represent a saturated linear or branched C1-C30 hydrocarbon radical, more particularly C1-C12, and in particular a methyl, ethyl, propyl or butyl radical.

More particularly R1 to R6 may be identical, and additionally may be a methyl radical.

With preference it is possible to have m=1 or 2 or 3, and/or n=0 and/or p=0 or 1.

The phenyl silicone oils may be selected from phenyltrimethicones, phenyldimethicones, phenyltrimethylsiloxydiphenylsiloxanes, diphenyldimethicones, diphenylmethyldiphenyltrisiloxanes, and mixtures thereof.

As a non-volatile phenyl silicone oil it is possible more particularly to use the phenyltrimethicones such as DC556 from Dow Corning (22.5 cSt), the oil Silbione 70663V30 from Rhone Poulenc (28 cSt), or the diphenyldimethicones such as the Belsil oils, more particularly Belsil PDM1000 (1000 cSt), Belsil PDM 200 (200 cSt) and Belsil PDM 20 (20 cSt) from Wacker. The values between parentheses represent the viscosities at 25° C.

The compatible oil may be selected from volatile oils.

For the purpose of the present invention, the term “volatile oil” is intended to mean an oil capable of evaporating on contact with keratin materials in less than one hour, at ambient temperature and atmospheric pressure. The volatile organic solvent(s) and the volatile oils of the invention are volatile cosmetic oils and organic solvents which are liquid at ambient temperature, and which have a non-zero vapour pressure at ambient temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg).

These oils can be hydrocarbon oils, silicone oils, fluoro oils, or mixtures thereof.

The term “hydrocarbon oil” is intended to mean an oil containing mainly hydrogen and carbon atoms and, optionally, oxygen, nitrogen, sulphur and phosphorus atoms. The volatile hydrocarbon oils can be chosen from hydrocarbon oils having from 8 to 16 carbon atoms, and in particular branched C₈-C₁₆ alkanes, such as C₈-C₁₆ isoalkanes of petroleum origin (also known as isoparaffins), for instance isododecane (also known as 2,2,4,4,6-pentamethylheptane), isodecane or isohexadecane, and, for example, the oils sold under the trade names Isopar or Permethyls, branched C₈-C₁₆ esters, isohexyl neopentanoate, and mixtures thereof. Other volatile hydrocarbon oils, such as petroleum distillates, in particular those sold under the name Shell Solt by the company Shell, can also be used. Preferably, the volatile solvent is chosen from volatile hydrocarbon oils having from 8 to 16 carbon atoms and mixtures thereof.

As volatile oils, use may also be made of volatile silicones, such as, for example, volatile linear or cyclic silicone oils, in particular those having a viscosity ≦8 centistokes (8×10⁻⁶ m²/s) and having in particular from 2 to 7 silicon atoms, these silicones optionally comprising alkyl or alkoxy groups having from 1 to 10 carbon atoms. As a volatile silicone oil that can be used in the invention, mention may in particular be made of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

Mention may also be made of the volatile linear alkyltrisiloxane oils of general formula (I):

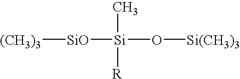

where R represents an alkyl group comprising from 2 to 4 carbon atoms, and one or more hydrogen atoms of which can be substituted with a fluorine or chlorine atom.

Among the oils of general formula (I), mention may be made of:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is respectively a butyl group, a propyl group or an ethyl group.

Use may also be made of volatile fluoro solvents such as nonafluoromethoxybutane or perfluoromethylcyclopentane.

The oil of formula (I) for which R is an ethyl group is more particularly sold under the name Baysilone TP 3886, and the oil for which R is a butyl group is more particularly sold under the name Baysilone TP 3887, by the company Bayer Silicones.

Preferably the compositions used each have a volatile oil content of less than or equal to 50% by weight, preferably less than or equal to 30% and more preferably less than or equal to 10% by weight relative to the total weight of each first and second composition. More preferably the compositions are free from volatile oil.

The compatible oil/compound X mass ratio is preferably between 1/10 and 5/1, preferably between 1/2 and 2/1.

The compatible oil/compound Y mass ratio is preferably between 1/10 and 5/1, preferably between 1/2 and 2/1.

### Compounds X and Y

The compounds X and Y can react together at a temperature between the ambient temperature and 180° C. Advantageously the compounds X and Y are able to react together at ambient temperature (20±5° C.) and atmospheric pressure, advantageously in the presence of a catalyst, by a hydrosilylation reaction or a condensation reaction or a crosslinking reaction in the presence of a peroxide.

Preferably these compounds X and Y are polymers. By polymers are meant compounds having at least two, preferably at least three, repeating units.

In one embodiment at least one of the compounds X and Y is a polymer whose main chain is formed predominantly of organosiloxane units.

In one particular embodiment the compounds X and the compounds Y are silicone compounds. A silicone compound is a compound comprising at least two organosiloxane units.

The compound or compounds X and the compound or compounds Y may be applied to the keratin materials via different compositions comprising the compound or compounds X and the compound or compounds Y, alone or in a mixture, or from a single composition comprising the compound or compounds X and the compound or compounds Y.

In one particular embodiment of the invention a composition (A) comprising the compound or compounds X, the compound or compounds Y and the compatible oil is applied to the keratin materials.

In another particular embodiment of the invention a composition (B) and a composition (C) comprising the compound or compounds X and the compound or compounds Y are applied to the keratin materials, the compatible oil being present in the composition (B) and/or the composition (C), and the order in which the compositions (B) and (C) are applied being arbitrary.

In another particular embodiment of the invention a composition (B), a composition (D) comprising the compound compounds X and a composition (E) comprising the compound or compounds Y are applied to the keratin materials, the compatible oil being present in the composition (B) and/or the composition (D) and/or the composition (E), and the order in which the compositions (B), (D) and (E) are applied being arbitrary.

In another particular embodiment of the invention a composition (F) comprising the compound or compounds X and a composition (E) comprising the compound or compounds Y are applied to the keratin materials, the compatible being present in the composition (F) and/or the composition (E), and the order in which the compositions (F) and (E) are applied being arbitrary.

In another particular embodiment of the invention at least one catalyst as defined above is applied to the keratin materials in order to activate the reaction between the compound or compounds X and the compound or compounds Y.

For example the catalyst or catalysts may be present in one or the other or in two or more of the compositions applied to the keratin materials, or in an additional composition, in which case the order in which the various compositions are applied to the keratin materials is arbitrary.

The silicone compounds X and Y mentioned below may have both film-forming properties and adhesive properties, depending, for example, on their proportion of silicone or on whether they are used in a mixture with a specific additive. It is possible, consequently, to modify the film-forming properties or the adhesive properties of such compounds in accordance with the intended use; this is the case in particular for the reactive elastomeric silicones known as “room temperature vulcanization” silicones.

### Polar Groups

In one particular embodiment one at least of the compounds X and Y carries at least one polar group able to form at least one hydrogen bond with the keratin materials. In one embodiment the compounds X and Y may be amine or non-amine compounds.

A polar group is a group which contains carbon atoms and hydrogen atoms in its chemical structure and also at least one heteroatom (such as O, N, S and P) such that said group is capable of establishing at least one hydrogen bond with the keratin materials.

Compounds which carry at least one group capable of establishing a hydrogen bond are particularly advantageous, since they provide the compositions containing them with improved adhesion to the keratin materials, owing to the capacity of these groups to establish a hydrogen bond with the keratin materials.

The polar group or groups carried by at least one of the compounds X and Y is or are capable of establishing a hydrogen bond, and include or includes either a hydrogen atom bonded to an electronegative atom. When the group contains a hydrogen atom bonded to an electronegative atom, the hydrogen atom can interact with another electronegative atom carried, for example, by another molecule, such as keratin, to form a hydrogen bond. When the group contains an electronegative atom, the electronegative atom can interact with a hydrogen atom bonded to an electronegative atom carried, for example, by another molecule, such as keratin, to form a hydrogen bond.

Advantageously these polar groups may be selected from the following groups:
carboxylic acids —COOH,
alcohols, such as —CH₂OH or —CH(R)OH, R being an alkyl radical containing 1 to 6 carbon atoms,
amino of formula —NR₁R₂, in which the radicals R₁ and R₂, which are identical or different, represent an alkyl radical containing 1 to 6 carbon atoms, or one of the radicals, R₁ or R₂, denotes a hydrogen atom,
pyridino,
amido of formula —NH—COR′ or —CO—NH—R′ in which R′ represents a hydrogen atom or an alkyl radical containing 1 to 6 carbon atoms,
pyrrolidino selected preferably from the groups of formula:

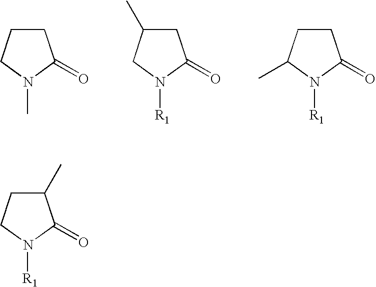

R₁ being an alkyl radical containing 1 to 6 carbon atoms,
carbamoyl of formula —O—CO—NH—R′ or —NH—CO—OR′, R′ being as defined above,
thiocarbamoyl, such as —O—CS—NH—R′ or —NH—CS—O—R′, R′ being as defined above,
ureyl such as —NR′—CO—N(R′)₂, the identical or different radicals R′ being as defined above,
sulphonamido such as —NR′—S(═O)₂—R′, R′ corresponding to the definition above.

Preferably these polar groups are present in an amount of less than or equal to 10% by weight relative to the weight of each compound X or Y, preferably less than or equal to 5% by weight, for example in an amount of from 1 to 3% by weight.

The polar group or groups may be situated in the main chain of the compound X and/or Y or are pendant to the main chain or are situated at the ends of the main chain of the compound X and/or Y.

### 1—Compounds X and Y Able to React by Hydrosilylation

In this embodiment the compounds X and Y are able to react by hydrosilylation, a reaction which can be depicted in a simplified way as follows:

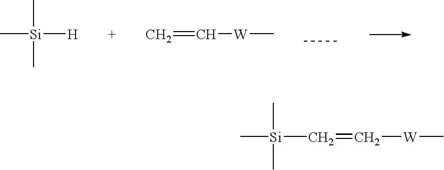

where W represents a carbon and/or silicone chain containing one or more unsaturated aliphatic groups.

In this case the compound X may be selected from silicone compounds comprising at least two unsaturated aliphatic groups. By way of example, the compound X may comprise a main silicone chain whose unsaturated aliphatic groups are pendant to the main chain (side group) or are situated at the ends of the main chain of the compound (terminal group). In the remainder of the description, these specific compounds will be referred to as polyorganosiloxanes having unsaturated aliphatic groups.

In one embodiment the compound X and/or the compound Y carries at least one polar group, as described above, able to form at least one hydrogen bond with the keratin materials. This polar group is advantageously carried by the compound X which comprises at least two unsaturated aliphatic groups.

In one embodiment the compound X is selected from polyorganosiloxanes comprising at least two unsaturated aliphatic groups, for example two or three vinyl or allyl groups, each bonded to a silicon atom.

In one advantageous embodiment the compound X is selected from polyorganosiloxanes containing siloxane units of formula:

$${R}_{m} {R}^{′} {SiO}_{\frac{( 3 - m )}{2}} \qquad ( I )$$

in which:
   R represents a monovalent linear or cyclic hydrocarbon group containing 1 to 30 carbon atoms, preferably 1 to 20, and more preferably 1 to 10 carbon atoms, such as, for example, a short-chain alkyl radical, containing for example 1 to 10 carbon atoms, in particular a methyl radical or else a phenyl group, preferably a methyl radical,
   m is 1 or 2, and
   R′ represents:
      an unsaturated aliphatic hydrocarbon group containing 2 to 10, preferably 2 to 5, carbon atoms such as, for example, a vinyl group or a group —R″—CH═CHR″ in which R″ is a divalent aliphatic hydrocarbon chain containing 1 to 8 carbon atoms which is bonded to the silicon atom, and R″′ is a hydrogen atom or an alkyl radical containing 1 to 4 carbon atoms, preferably a hydrogen atom; possibilities for group R′ include vinyl groups, allyl groups and mixtures thereof; or
      an unsaturated cyclic hydrocarbon group containing 5 to 8 carbon atoms, such as, for example, a cyclohexenyl group.
   Preferably, R′ is an unsaturated aliphatic hydrocarbon group, preferably a vinyl group.

In one particular embodiment the polyorganosiloxane further comprises units of formula

$${R}_{n} {SiO}_{\frac{( 4 - n )}{2}} \qquad ( II )$$

in which R is a group as defined above and n is 1, 2 or 3.

In one version the compound X comprises a silicone resin containing at least two ethylenic unsaturations, said resin being capable of reacting with the compound Y by hydrosilylation. Possible examples include the resins of type MQ or MT which themselves carry unsaturated reactive end groups —CH═CH₂.

These resins are crosslinked organosiloxane polymers.

The nomenclature of silicone resins is known by the name of “MDTQ”, the resin being described as a function of the different monomeric siloxane units it comprises, with each of the letters MDTQ characterizing one type of unit.

The letter M represents the monofunctional unit of formula (CH₃)₃SiO_{1/2}, the silicon atom being joined to a single oxygen atom in the polymer comprising this unit. The letter D signifies a difunctional unit (CH₃)₂SiO_{2/2} in which the silicon atom is joined to two oxygen atoms.

The letter T represents a trifunctional unit of formula (CH₃)SiO_{3/2}.

In the units M, D and T defined above, at least one of the methyl groups may be substituted by a group R other than the methyl group, such as a hydrocarbon radical (more particularly alkyl) having 2 to 10 carbon atoms, or a phenyl group, or else a hydroxyl group.

Finally, the letter Q signifies a tetrafunctional unit SiO_{4/2} in which the silicon atom is bonded to four hydrogen atoms which are themselves bonded to the remainder of the polymer. Possible examples of such resins include the MT silicone resins such as the poly(phenylvinylsilsesquioxanes) like those sold under the name SST-3PV1 by Gelest.

Advantageously the compound X is selected from polyorganopolysiloxanes, more particularly those comprising the siloxane units (I) and optionally (II) described above.

The compound Y preferably comprises at least two free Si—H groups (hydrogenosilane groups).

The compound Y may advantageously be selected from organosiloxanes comprising at least one alkylhydrogenosiloxane unit of formula:

$${R}_{p} {HSiO}_{\frac{( 3 - p )}{2}} \qquad ( III )$$

in which:
R represents a monovalent linear or cyclic hydrocarbon group containing 1 to 30 carbon atoms, such as, for example, an alkyl radical having 1 to 30 carbon atoms, preferably 1 to 20 and more preferably 1 to 10 carbon atoms, in particular a methyl radical, or else a phenyl group, and p is 1 or 2. Preferably R is a hydrocarbon group, preferably methyl.

These organosiloxane compounds Y having alkylhydrogenosiloxane units may further comprise units of formula:

$${R}_{n} {SiO}_{\frac{( 4 - n )}{2}} \qquad ( II )$$

as defined above.

The compound Y may be a silicone resin comprising at least one unit selected from the M, D, T and Q units as defined above and comprising at least one Si—H group, such as the poly(methylhydridosilsesquioxanes) sold under the name SST-3 MH1.1 by Gelest.

Preferably these organosiloxane compounds Y contain from 0.5% to 2.5% by weight of Si—H groups.

Advantageously the radicals R represent a methyl group in the formulae above.

Preferably these organosiloxanes Y comprise terminal groups of formula CH₃SiO_{1/2}.

Advantageously the organosiloxanes Y comprise at least two alkylhydrogenosiloxane units of formula H₃CHSiO and optionally comprise units (H₃C)₂SiO.

Organosiloxane compounds Y of this kind containing hydrogenosilane groups are described for example in document EP 0465744.

In one version the compound X is selected from organic oligomers or polymers (organic compounds are those whose main chain is not a silicone chain, preference being given to compounds containing no silicon atoms) or from hybrid organic/silicone polymers or oligomers, said oligomers or polymers carrying at least two reactive unsaturated aliphatic groups, and the compound Y being selected from the aforementioned hydrogenosiloxanes.

In one embodiment the organic or organic/silicone hybrid compounds X carrying at least two reactive unsaturated aliphatic groups carry at least one polar group as described above.

The compound X, which is organic in nature, may then be selected from vinyl and (meth)acrylic oligomers or polymers, polyesters, polyurethanes and/or polyureas, polyethers, perfluoropolyethers, polyolefins such as polybutene and polyisobutylene, dendrimers or hyperbranched organic polymers, or mixtures thereof.

In particular the organic polymer or the organic part of the hybrid polymer may be selected from the following polymers:
a) ethylenically unsaturated polyesters:
   this is a group of polymers of polyester type having at least 2 ethylenic double bonds distributed anywhere in the main chain of the polymer. These unsaturated polyesters are obtained by polycondensation of a mixture:
      of linear or branched aliphatic or cycloaliphatic carboxylic diacids containing in particular 3 to 50 carbon atoms, preferably 3 to 20 and more preferably 3 to 10 carbon atoms, such as adipic acid or sebacic acid, aromatic carboxylic diacids having in particular 8 to 50 carbon atoms, preferably 8 to 20 and more preferably 8 to 14 carbon atoms, such as phthalic acids, more particularly terephthalic acid, and/or carboxylic diacids obtained from dimers of ethylenically unsaturated fatty acids, such as the dimers of oleic or linoleic acids that are described in patent application EP-A-959 066 (paragraph [0021]) and are sold under the name Pripol® by Unichema or Empol® by Henkel, all of these diacids necessarily being devoid of polymerizable ethylenic double bonds,
      of linear or branched aliphatic or cycloaliphatic diols containing in particular 2 to 50 carbon atoms, preferably 2 to 20 and more preferably 2 to 10 carbon atoms, such as ethylene glycol, diethylene glycol, propylene glycol, 1,4-butanediol or cyclohexanedimethanol, aromatic diols having 6 to 50 carbon atoms, preferably 6 to 20 and more preferably 6 to 15 carbon atoms, such as bisphenol A and bisphenol B, and/or diol dimers obtained from the reduction of dimers of fatty acids as defined above, and
      of one or more carboxylic diacids or their anhydrides containing at least one polymerizable ethylenic double bond and having 3 to 50 carbon atoms, preferably 3 to 20 and more preferably 3 to 10 carbon atoms, such as maleic acid, fumaric acid or itaconic acid.
b) Polyesters having side and/or terminal (meth)acrylate groups:
   this is a group of polymers of polyester type which are obtained by polycondensation of a mixture:
      of linear or branched aliphatic or cycloaliphatic carboxylic diacids containing in particular 3 to 50 carbon atoms, preferably 3 to 20 and more preferably 3 to 10 carbon atoms, such as adipic acid or sebacic acid, aromatic carboxylic diacids having in particular 8 to 50 carbon atoms, preferably 8 to 20 and more preferably 8 to 14 carbon atoms, such as phthalic acids, more particularly terephthalic acid, and/or carboxylic diacids obtained from dimers of ethylenically unsaturated fatty acids, such as the dimers of oleic or linoleic acids that are described in patent application EP-A-959 066 (paragraph [0021]) and are sold under the name Pripol® by Unichema or Empol® by Henkel, all of these diacids necessarily being devoid of polymerizable ethylenic double bonds,
      of linear or branched aliphatic or cycloaliphatic diols containing in particular 2 to 50 carbon atoms, preferably 2 to 20 and more preferably 2 to 10 carbon atoms, such as ethylene glycol, diethylene glycol, propylene glycol, 1,4-butanediol or cyclohexanedimethanol, aromatic diols having 6 to 50 carbon atoms, preferably 6 to 20 and more preferably 6 to 15 carbon atoms, such as bisphenol A and bisphenol B, and
      of at least one ester of (meth)acrylic acid and a diol or polyol having 2 to 20 carbon atoms, preferably 2 to 6 carbon atoms, such as 2-hydroxyethyl (meth)acrylate, 2-hydroxypropyl (meth)acrylate and glycerol methacrylate.
   These polyesters are different from those described above in section a) in that the ethylenic double bonds are situated not in the main chain but on side groups or at the end of the chains. These ethylenic double bonds are those of the (meth)acrylate groups present in the polymer.
   Polyesters of this kind are sold for example by UCB under the name Ebecryl® (Ebecryl® 450: molar mass 1600, on average 6 acrylate functions per molecule, Ebecryl® 652: molar mass 1500, on average 6 acrylate functions per molecule, Ebecryl® 800: molar mass 780, on average 4 acrylate functions per molecule, Ebecryl® 810: molar mass 1000, on average 4 acrylate functions per molecule, Ebecryl® 50 000: molar mass 1500, on average 6 acrylate functions per molecule).
c) Polyurethanes and/or polyureas having (meth)acrylate groups, obtained by polycondensation:
   of aliphatic, cycloaliphatic and/or aromatic diisocyanates, triisocyanates and/or polyisocyanates, having in particular 4 to 50, preferably 4 to 30, carbon atoms, such as hexamethylene diisocyanate, isophorone diisocyanate, toluene diisocyanate, diphenylmethane diisocyanate or the isocyanurates of formula:

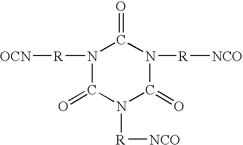

resulting from the trimerization of 3 molecules of diisocyanates OCN—R—CNO, where R is a linear, branched or cyclic hydrocarbon radical containing 2 to 30 carbon atoms,
of polyols, more particularly of diols, which are devoid of polymerizable ethylenic unsaturations, such as 1,4-butanediol, ethylene glycol or trimethylolpropane, and/or from polyamines, more particularly diamines, which are aliphatic, cycloaliphatic and/or aromatic and have more particularly 3 to 50 carbon atoms, such as ethylenediamine or hexamethylenediamine, and
from at least one ester of (meth)acrylic acid and a diol or polyol having 2 to 20 carbon atoms, preferably 2 to 6 carbon atoms, such as 2-hydroxyethyl (meth)-acrylate, 2-hydroxypropyl (meth)acrylate and glycerol methacrylate.
Polyurethanes/polyureas of these kinds containing acrylate groups are sold for example under the name SR 368 (tris(2-hydroxyethyl) isocyanurate triacrylate) or Craynor® 435 by Cray Valley, or under the name Ebecryl® by UCB (Ebecryl® 210: molar mass 1500, 2 acrylate functions per molecule, Ebecryl® 230: molar mass 5000, 2 acrylate functions per molecule, Ebecryl® 270: molar mass 1500, 2 acrylate functions per molecule, Ebecryl® 8402: molar mass 1000, 2 acrylate functions per molecule, Ebecryl® 8804: molar mass 1300, 2 acrylate functions per molecule, Ebecryl® 220: molar mass 1000, 6 acrylate functions per molecule, Ebecryl® 2220: molar mass 1200, 6 acrylate functions per molecule, Ebecryl® 1290: molar mass 1000, 6 acrylate functions per molecule, Ebecryl® 800: molar mass 800, 6 acrylate functions per molecule).
Mention may also be made of the water-soluble aliphatic polyurethane diacrylates sold under the names Ebecryl® 2000, Ebecryl® 2001 and Ebecryl® 2002, and of the polyurethane diacrylates in aqueous dispersion that are sold under the trade names IRR® 390, IRR® 400, IRR® 422 and IRR® 424 by UCB.
d) Polyethers having (meth)acrylate groups which are obtained by esterification, by (meth)-acrylic acid, of terminal hydroxyl groups of homopolymers or copolymers of C₁₋₄ alkylene glycols, such as polyethylene glycol, polypropylene glycol, the copolymers of ethylene oxide and propylene oxide having preferably a weight-average molecular mass of less than 10 000, and polyethoxylated or polypropoxylated trimethylolpropane. Polyoxyethylene di(meth)acrylates of appropriate molar mass are sold for example under the names SR 259, SR 344, SR 610, SR 210, SR 603 and SR 252 by Cray Valley or under the name Ebecryl® 11 by UCB. Polyethoxylated trimethylolpropane triacrylates are sold for example under the names SR 454, SR 498, SR 502, SR 9035 and SR 415 by Cray Valley or under the name Ebecryl® 160 by UCB. Polypropoxylated trimethylolpropane triacrylates are sold for example under the names SR 492 and SR 501 by Cray Valley.
e) Epoxy acrylates obtained by reacting
   at least one diepoxide selected for example from:
      (i) bisphenol A diglycidyl ether,
      (ii) a diepoxy resin resulting from the reaction of bisphenol A diglycidyl ether and epichlorohydrin,
      (iii) an epoxy ester resin having α,ω-diepoxy ends, resulting from the condensation of a dicarboxylic acid having 3 to 50 carbon atoms with a stoichiometric excess of (i) and/or (ii),
      (iv) an epoxy ether resin having α,ω-diepoxy ends, resulting from the condensation of a diol having 3 to 50 carbon atoms with a stoichiometric excess of (i) and/or (ii),
      (v) natural or synthetic oils carrying at least 2 epoxide groups, such as epoxidized soya oil, epoxidized linseed oil and epoxidized vernonia oil,
      (vi) a phenol-formaldehyde polycondensate (Novolac® resin) whose ends and/or side groups have been epoxidized,
      and
      one or more carboxylic acids or polycarboxylic acids containing at least one ethylenic double bond positioned α,β to the carboxyl group, such as (meth)acrylic acid or crotonic acid, or the esters of (meth)acrylic acid and a diol or polyol having 2 to 20 carbon atoms, preferably 2 to 6 carbon atoms, such as 2-hydroxyethyl (meth)acrylate.
Polymers of this kind are sold for example under the names SR 349, SR 601, CD 541, SR 602, SR 9036, SR 348, CD 540, SR 480 and CD 9038 by Cray Valley, under the names Ebecryl® 600 and Ebecryl® 609, Ebecryl® 150, Ebecryl® 860 and Ebecryl® 3702 by UCB, and under the names Photomer® 3005 and Photomer® 3082 by Henkel.
f) Poly-C₁₋₅₀ alkyl (meth)acrylates, said alkyl being linear, branched or cyclic, containing at least two functions having an ethylenic double bond, which are carried by the terminal and/or side hydrocarbon chains.
Copolymers of this kind are sold for example under the names IRR® 375, OTA® 480 and Ebecryl® 2047 by UCB.
g) Polyolefins such as polybutene and polyisobutylene.
h) Perfluoropolyethers having acrylate groups which are obtained by esterification, for example with (meth)acrylic acid, of perfluoropolyethers which carry terminal and/or side hydroxyl groups.
α,ω-Diol perfluoropolyethers of this kind are described in particular in EP-A-1057849 and are sold by Ausimont under the name Fomblin® Z DIOL.
i) Dendrimers and hyperbranched polymers which carry terminal (meth)acrylate or (meth)-acrylamide groups obtained respectively by esterification or amidification of dendrimers and hyperbranched polymers having terminal hydroxyl or amino functions with (meth)acrylic acid.
Dendrimers (from the Greek dendron=tree) are “arborescent”, in other words highly branched, polymer molecules invented by D. A. Tomalia and his team at the beginning of the 1990s (Donald A. Tomalia et al., Angewandte Chemie, Int. Engl. Ed., vol. 29, no. 2, pages 138-175). They are structures constructed around a generally polyfunctional central unit. Arrayed in chains around this central unit, in accordance with a well-defined structure, are branched chain-extension units, hence giving rise to monodisperse symmetrical macromolecules which have a well-defined chemical and stereochemical structure. Polyamidoamine dendrimers are sold for example under the name Starburst® by Dendritech.
Hyperbranched polymers are polycondensates, generally of polyester, polyamide or polyethyleneamine type, which are obtained from polyfunctional monomers, which have an arborescent structure similar to that of the dendrimers but much less regular than them (see, for example, WO-A-93/17060 and WO 96/12754).
Under the name Boltorn®, the company Perstorp sells hyperbranched polyesters. Hyperbranched polyethylene amines are found under the name Comburst® from the company Dendritech. Hyperbranched poly(esteramides) having hydroxyl ends are sold by the company DSM under the name Hybrane®.
These dendrimers and hyperbranched polymers esterified or amidified by acrylic and/or methacrylic acid differ from the polymers described in sections a) to h) above in the very large number of ethylenic double bonds present. This high functionality, most often greater than 5, makes them particularly useful, allowing them to act as a “crosslinking node”, in other words as a site of multiple crosslinking.
It is therefore possible to use these dendritic and hyperbranched polymers in combination with one or more of the polymers and/or oligomers a) to h) above.

### 1a Additional Reactive Compounds

In one embodiment the compositions comprising the compound X and/or Y may further comprise at least one additional reactive compound such as:
organic or inorganic particles comprising on their surface at least 2 unsaturated aliphatic groups, examples including silicas surface-treated with, for example, silicone compounds having vinyl groups, such as, for example, cyclotetramethyltetravinylsiloxane-treated silica;
silazane compounds such as hexamethyldisilazane.

### 1b Catalyst

The hydrosilylation reaction takes place advantageously in the presence of a catalyst which may be present in one or other of the compositions comprising X and/or Y or in a separate composition, the catalyst being preferably based on platinum or on tin.

Examples include catalysts based on platinum deposited on a silica gel support or on a charcoal powder support, platinum chloride, platinum salts and chloroplatinic acids.

Preference is given to using chloroplatinic acids in hexahydrate or anhydrous form, which are readily dispersible in organosilicone media.

Mention may also be made of platinum complexes such as those based on chloroplatinic acid hexahydrate and divinyltetramethyldisiloxane.

The catalyst may be present in one or other of the compositions useful in the present invention in an amount of from 0.0001% to 20% by weight relative to the total weight of the composition comprising it.

In the compositions of the invention it is also possible to introduce polymerization inhibitors or retardants, and more particularly catalyst inhibitors, for the purpose of increasing the stability of the composition over time or of retarding the polymerization. Without limitation mention may be made of cyclic polymethylvinylsiloxanes, and especially tetravinyltetramethylcyclotetrasiloxane, and acetylenic alcohols, preferably volatile acetylenic alcohols, such as methylisobutynol.

The presence of ionic salts, such as sodium acetate, in one and/or the other of the first and second compositions may influence the rate of polymerization of the compounds.

An example of such compounds X and Y which react by hydrosilylation includes the following references provided by Dow Corning: DC 7-9800 Soft Skin Adhesive Parts A & B.

Advantageously the compounds X and Y are selected from silicone compounds able to react by hydrosilylation; in particular, the compound X is selected from polyorganosiloxanes containing units of formula (I) described above, and the compound Y is selected from organosiloxanes containing alkylhydrogenosiloxane units of formula (III) described above. In one particular embodiment the compound X is a polydimethylsiloxane having terminal vinyl groups, and the compound Y is a methylhydrogenosiloxane.

By way of example, the two following mixtures X′ and Y′ are used, containing the compounds X and Y. These mixtures may be supplied by Dow Corning.

MIXTURE X′:

| Ingredient (INCI name) | CAS No. | Amounts (%) | Function |
|---|---|---|---|
| Dimethyl Siloxane, | 68083-19-2 | 55-95 | polymer |
| Dimethylvinylsiloxy- | | | |
| terminated | | | |
| Silica Silylate | 68909-20-6 | 10-40 | filler |
| 1,3-Diethenyl-1,1,3,3- | 68478-92-2 | trace | catalyst |
| tetramethyldisiloxane | | | |
| complex | | | |
| Tetramethyldivinyl- | 2627-95-4 | 0.1-1 | polymer |
| disiloxane | | | |

MIXTURE Y′

| Ingredient (INCI name) | CAS No. | Amounts (%) | Function |
|---|---|---|---|
| Dimethyl Siloxane, | 68083-19-2 | 55-95 | polymer |
| Dimethylvinylsiloxy | | | |
| Silica Silylate | 68909-20-6 | 10-40 | filler |
| Dimethyl, Methyl- | 68037-59-2 | 1-10 | polymer |
| hydrogeno Siloxane, | | | |
| trimethylsiloxy- | | | |
| terminated | | | |

### 2/ Compounds X and Y Able to React by Condensation

In this embodiment the compounds X and Y are able to react by condensation, either in the presence of water (hydrolysis), by reaction of 2 compounds which carry alkoxysilane groups, or by so-called direct condensation, by reaction of a compound which carries one or more alkoxysilane groups and a compound which carries one or more silanol groups, or by reaction of 2 compounds which carry one or more silanol groups.

When the condensation takes place in the presence of water, the water may be in particular ambient moisture, residual water on the skin, the lips, the lashes and/or the nails, or water provided by an external source, for example by wetting of the lashes beforehand (for example by means of an atomizer, natural or artificial tears).

In this mode of condensation reaction, the compounds X and Y, which are identical or different, may therefore be selected from silicone compounds whose main chain comprises at least two alkoxysilane groups and/or at least two silanol (Si—OH) groups, which are side groups and/or chain-end groups.

In one embodiment, the compound X and/or the compound Y carries at least one polar group, as described above, capable of forming at least one hydrogen bond with the keratin materials.

In one advantageous embodiment the compounds X and/or Y are selected from polyorganosiloxanes comprising at least two alkoxysilane groups. An alkoxysilane group is a group comprising at least one moiety —Si—OR, R being an alkyl group containing 1 to 6 carbon atoms.

The compounds X and Y are more particularly selected from polyorganosiloxanes comprising terminal alkoxy-silane groups, more specifically those which comprise at least 2 terminal alkoxysilane groups, preferably terminal trialkoxysilane groups.

These compounds X and/or Y preferably comprise predominantly units of formula

R⁹_{S}SiO_{(4-s)/2}. (IV)

in which the R⁹ independently represent a radical selected from alkyl groups containing 1 to 6 carbon atoms, phenyl, and fluoroalkyl groups, and s is 0, 1, 2 or 3. Preferably the R⁹ independently represent alkyl groups containing 1 to 6 carbon atoms. As the alkyl group, mention may be made in particular of methyl, propyl, butyl, hexyl and mixtures thereof, preferably methyl or ethyl. As a fluoroalkyl group, mention may be made of 3,3,3-trifluoropropyl.

In one particular embodiment the compounds X and Y, which are identical or different, are polyorganosiloxanes comprising units of formula

(R⁹₂SiO₂)_{f}. (V)

in which R⁹ is as described above, R⁹ preferably being a methyl radical, and f is such that the polymer has a viscosity at 25° C. of from 0.5 to 3000 Pa·s, preferably of from 5 to 150 Pa·s. For example f may be from 2 to 5000, preferably from 3 to 3000, more preferably from 5 to 1000.

These polyorganosiloxane compounds X and Y comprise at least two terminal trialkoxysilane groups per polymer molecule, said groups having the formula

ZSiR¹_{X}(OR)₃₋ₓ, (VI)

in which
the radicals R represent independently a methyl, ethyl, n-propyl, isopropyl, n-butyl, sec-butyl or isobutyl group, preferably a methyl or ethyl group,
R¹ is a methyl or ethyl group,
x is 0 or 1, preferably 0 and
Z is selected from the following: divalent hydrocarbon groups containing no ethylenic unsaturation and containing 1 to 18 carbon atoms, preferably 2 to 18 carbon atoms (alkylene groups), the combinations of divalent hydrocarbon radicals and of siloxane segments of formula:

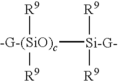

R⁹ being as described above, G is a divalent hydrocarbon radical containing no ethylenic unsaturation and containing 1 to 18 carbon atoms, preferably 2 to 18 carbon atoms, and c is an integer of from 1 to 6.

Z and G may more particularly be selected from alkylene groups such as methylene, ethylene, propylene, butylene, pentylene and hexylene, and arylene groups such as phenylene.

Preferably Z is an alkylene group, and more preferably ethylene.

These polymers may have on average at least 1.2 trialkoxysilane end groups or terminal trialkoxysilane chains per molecule, and preferably on average at least 1.5 trialkoxysilane end groups per molecule. These polymers may have at least 1.2 trialkoxysilane end groups per molecule, and some may comprise other types of end groups, such as end groups of formula CH₂═CH—SiR⁹₂— or of formula R⁶₃—Si—, in which R⁹ is as defined above and each group R⁶ is selected independently from groups R⁹ or vinyl. Possible examples of such end groups include trimethoxysilane, triethoxysilane, vinyldimethoxysilane and vinylmethyloxyphenylsilane groups.

Polymers of this kind are described more particularly in documents U.S. Pat. No. 3,175,993, U.S. Pat. No. 4,772,675, U.S. Pat. No. 4,871,827, U.S. Pat. No. 4,888,380, U.S. Pat. No. 4,898,910, U.S. Pat. No. 4,906,719 and U.S. Pat. No. 4,962,174, whose content is incorporated by reference into the present patent application.

As compound X and/or Y it is possible in particular to mention the polymer of formula

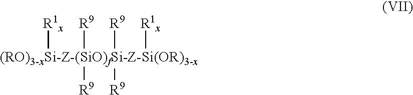

in which R, R¹, R⁹, Z, x and f are as described above.

The compounds X and/or Y may further comprise a mixture of polymer of formula (VII) above with polymers of formula (VIII):

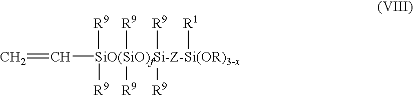

in which R, R¹, R⁹, Z, x and f are as described above. When the polyorganosiloxane compound X and/or Y having one or more alkoxysilane groups comprises such a mixture, the different polyorganosiloxanes are present in amounts such that the terminal organosilyl chains represent less than 40%, preferably less than 25%, by number of the terminal chains.

The particularly preferred polyorganosiloxane compounds X and/or Y are those of formula (VII) that were described above. Compounds X and/or Y of this kind are described for example in document WO 01/96450.

As indicated above, the compounds X and Y may be identical or different.

In one version one of the two reactive compounds, X or Y, is of silicone type and the other is of organic type. For example, the compound X is selected from organic oligomers or polymers or organic/silicone hybrid oligomers or polymers, said polymers or oligomers comprising at least two alkoxysilane groups, and Y is selected from silicone compounds such as the polyorganosiloxanes described above. In particular the organic oligomers or polymers are selected from vinyl and (meth)acrylic oligomers or polymers, polyesters, polyamides, polyurethanes and/or polyureas, polyethers, polyolefins, perfluoropolyethers, organic dendrimers and hyperbranched polymers, and mixtures thereof.

The organic polymers of vinyl or (meth)acrylic kind which carry alkoxysilane side groups may in particular be obtained by copolymerizing at least one vinyl or (meth)acrylic organic monomer with a (meth)acryloyloxypropyltrimethoxysilane, a vinyltrimethoxysilane, a vinyltriethoxysilane, an allyltrimethoxysilane, etc. Mention may be made for example of the (meth)acrylic polymers described in the document of Kusabe. M, Pitture e Vernici—European Coating; 12-B, pages 43-49, 2005, and more particularly the polyacrylates having alkoxysilane groups that are called MAX, from Kaneka, or those described in the publication of Probster, M, Adhesion-Kleben & Dichten, 2004, 481 (1-2), pages 12-14.

The organic polymers which result from a polycondensation or from a polyaddition, such as polyesters, polyamides, polyurethanes and/or polyureas, and polyethers, and which carry alkoxysilane side and/or end groups, may result, for example, from the reaction of an oligomeric prepolymer as described above with one of the following silane coreactants which carry at least one alkoxysilane group: aminopropyltrimethoxysilane, aminopropyltriethoxysilane, aminoethylaminopropyltrimethoxysilane, glycidyloxypropyltrimethoxysilane, glycidyloxypropyltriethoxysilane, epoxycyclohexylethyltrimethoxysilane, mercaptopropyltrimethoxysilane.

Examples of polyethers and of polyisobutylenes having alkoxysilane groups are described in the publication of Kusabe, M., Pitture e Vernici—European Coating; 12-B, pages 43-49, 2005. Possible examples of polyurethanes having alkoxysilane end groups are those described in the document of Probster, M., Adhesion-Kleben & Dichten, 2004, 481 (1-2), pages 12-14 or else those described in the document of Landon, S., Pitture e Vernici vol. 73, No. 11, pages 18-24, 1997 or in the document of Huang, Mowo, Pitture e Vernici vol. 5, 2000, pages 61-67; mention may be made in particular of the polyurethanes having alkoxysilane groups from OSI-WITCO-GE.

As polyorganosiloxane compounds X and/or Y, mention may be made of the resins of type MQ or MT which themselves carry alkoxysilane and/or silanol ends, such as, for example, the poly(isobutylsilsesquioxane) resins functionalized with silanol groups that are provided under the name SST-S7C41 (3 Si—OH groups) by Gelest.

### 2a Additional Reactive Compound

One of the compositions useful in the present invention may further comprise an additional reactive compound comprising at least two alkoxysilane or silanol groups. Possible examples include:
one or more organic or inorganic particles comprising on their surface alkoxysilane and/or silanol groups, for example fillers surface-treated with such groups.

### 2b Catalyst

The condensation reaction may take place in the presence of a metal-based catalyst, which may be present in one or the other of the compositions comprising X and/or Y or in a separate composition. The catalyst useful in this type of reaction is preferably a catalyst based on titanium.

Mention may be made in particular of the tetraalkoxytitanium-based catalysts of formula

Ti(OR²)_{y}(OR³)_{4-y},

in which R² is selected from tertiary alkyl radicals such as tert-butyl, tert-amyl and 2,4-dimethyl-3-pentyl; R³ represents an alkyl radical containing 1 to 6 carbon atoms, preferably a methyl, ethyl, n-propyl, isopropyl, n-butyl, sec-butyl or hexyl group; and y is a number of from 3 to 4, more preferably from 3.4 to 4.

The catalyst may be present in the one or the other of the compositions useful in the present invention, in an amount of from 0.0001% to 20% by weight relative to the total weight of the composition or compositions containing it.

### 2c Diluent

The useful compositions comprising X and/or Y may further comprise a volatile silicone oil (or diluent) intended for lowering the viscosity of the composition. This oil may be selected from short-chain linear silicones such as hexamethyldisiloxane and octamethyltrisiloxane and from cyclic silicones such as octamethylcyclotetrasiloxane and decamethylcyclopentasiloxane, and mixtures thereof.

This silicone oil may represent from 5% to 95%, preferably from 10% to 80%, by weight relative to the weight of each composition.

As an example of compounds X and Y which carry alkoxysilane groups and react by condensation, mention may be made of the following mixtures X′ and Z′, prepared by Dow Corning:

Mixture X′:

| Ingredient (INCI name) | CAS No. | Amounts (%) | Function |
|---|---|---|---|
| Bis-Trimethoxysiloxy- | PMN87176 | 25-45 | polymer |
| ethyl Tetramethyldi- | | | |
| siloxyethyl | | | |
| Dimethicone (1) | | | |
| Silica Silylate | 68909-20-6 | 5-20 | filler |
| Disiloxane | 107-46-0 | 30-70 | solvent |

(1) The mixture X′ contains identical compounds X and Y

Mixture Z′:

| Ingredient (INCI name) | CAS No. | Amounts (%) | Function |
|---|---|---|---|
| Disiloxane | 107-46-0 | 80-99 | solvent |
| Tetra T Butyl Titanate | — | 1-20 | catalyst |

### 3/ Crosslinking in the Presence of Peroxide

In one embodiment the invention provides a make-up kit comprising at least two compositions which are packaged separately,
the kit comprising at least one compound X, at least one compound Y and at least one peroxide, at least one of the compounds, X or Y, being a silicone compound,
with the proviso that the compounds X and Y—and the peroxide, are not simultaneously in one of said compositions,
said compounds X and Y reacting together by a crosslinking reaction in the presence of a peroxide, when they are brought into contact with one another,
one at least of the first and second compositions comprising at least one compatible oil,
said compounds X and Y and said compatible oil, and their respective amounts, being such that the transfer index of a deposit comprising the two compositions is less than or equal to 40 out of 100.

This reaction takes place preferably by heating at a temperature greater than or equal to 50° C., preferably greater than or equal to 80° C., and of up to 120° C.

In this case the compounds X and Y, which are identical or different, comprise at least two —CH₃ side groups and/or at least two side chains which carry a —CH₃ group.

The compounds X and Y are preferably silicone compounds and may be selected, for example, from non-volatile linear polydimethylsiloxanes of high molecular weight, having a degree of polymerization of more than 6, which have at least two —CH₃ side groups joined to the silicon atom and/or at least two side chains which carry a —CH₃ group. Examples include the polymers described in the “Reactive Silicones” catalogue of the company Gelest Inc., 2004 edition, page 6, and more particularly the vinylmethylsiloxane-dimethylsiloxane copolymers (also called rubbers) of molecular weights of from 500 000 to 900 000 and in particular of a viscosity greater than 2 000 000 cSt.

As peroxides which can be used in the context of the invention, mention may be made of benzoyl peroxide, 2,4-dichlorobenzoyl peroxide and mixtures thereof.

In one embodiment the hydrosilylation reaction, the condensation reaction or the crosslinking reaction in the presence of a peroxide between the compounds X and Y is accelerated by provision of heat, with the temperature of the system being raised, for example, to between 25° C. and 180° C. The system will react more particularly on the skin.

Generally speaking, irrespective of the type of reaction by which the compounds X and Y react together, the molar percentage of X relative to the entirety of the compounds X and Y, i.e. the ratio X/(X+Y)×100, may range from 5% to 95%, preferably from 10% to 90%, more preferably still from 20% to 80%.

Similarly, the molar percentage of Y relative to the entirety of the compounds X and Y, i.e. the ratio Y/(X+Y)×100, may range from 5% to 95%, preferably from 10% to 90%, more preferably still from 20% to 80%.

The compound X may have a weight-average molecular mass (Mw) of from 150 to 1 000 000, preferably from 200 to 800 000, more preferably from 200 to 250 000.

The compound Y may have a weight-average molecular mass (Mw) of from 200 to 1 000 000, preferably from 300 to 800 000, more preferably from 500 to 250 000.

The compound X may represent from 0.5% to 95% by weight relative to the total weight of the useful compositions or relative to the total weight of the composition when X and Y are present within the same composition, preferably from 1% to 90% by weight, and more preferably from 5% to 80% by weight.

The compound Y may represent from 0.05% to 95% by weight relative to the total weight of the useful compositions or relative to the total weight of the composition when X and Y are present within the same composition, preferably from 1% to 90% by weight, and more preferably from 5% to 80% by weight.

The ratio between the compounds X and Y may be changed so as to modify the rate of reaction and hence the rate at which the film is formed, or else so as to adapt the properties of the resulting film (for example its adhesive properties) in accordance with the desired application.

One at least of the compositions forming the kit may comprise fillers. By way of example, these fillers may be colloidal calcium carbonate, which may be treated or untreated with stearic acid or stearate, or silica such as fumed silicas and precipitated silicas, hydrophobically treated silicas, ground quartz, alumina, aluminium hydroxide, titanium dioxide, diatomaceous earth, iron oxide, carbon black, and graphite. Synthetic silicas whose surface is modified with silica compounds to make them superficially hydrophobic are particularly preferred. These fillers are different from one another in their surface properties, in the silicone compounds used to treat the silica, and in the way in which the surface treatment is conducted. Fillers of this kind make it possible to reduce the viscosity of the formulation obtained from the compounds X and/or Y. Moreover, resin-based reinforcing fillers may also be used. Preference as a filler is given to silica, calcium carbonate, and resin-based fillers.

Possible examples include the treated fillers Cab-O-Sil®TS-530, Aerosil®R8200 and Wacker HDX H2000.

### Non-Transfer of the Deposit

The transfer index
of a film obtained by depositing a composition comprising the compounds X and Y and the compatible oil, or
of a film obtained by successive depositions of a composition comprising the compound X and of a composition comprising the compound Y, one at least of the two compositions comprising a compatible oil,
   is preferably less than or equal to 40 out of 100.

More preferably the transfer index is less than or equal to 30, preferably less than or equal to 20, more preferably less than or equal to 15, preferably less than or equal to 10, preferably less than or equal to 5 out of 100, and preferably less than or equal to 2 out of 100.

The transfer index may be measured according to the following method.

A substrate (rectangle of 40 mm×70 mm and of thickness 3 mm) of polyethylene foam that is adhesive on one of the faces, having a density of 33 kg/m³ (sold under the name RE40X70EP3 from the company Joint Technique Lyonnais Ind.) is preheated on a hotplate maintained at a temperature of 40° C. so that the surface of the substrate is maintained at a temperature of 33° C.±1° C.

While the substrate is left on the hotplate, the composition or mixture of the two compositions is applied to the whole non-adhesive surface of the substrate, by spreading it using a fine brush, to give a deposit of the composition of approximately 15 μm, and then it is left to dry for 30 minutes.

After drying, the substrate is bonded by its adhesive face to an anvil with a diameter of 20 mm which is equipped with a screw pitch. The substrate/deposit assembly is then cut out using a punch 18 mm in diameter. The anvil is subsequently screwed onto a press (Statif Manuel Imada SV-2 from the company Someco) equipped with a dynamometer (Imada DPS-20 from the company Someco).

A piece of 80 g/m² white photocopier paper is placed on the bed of the press and then the substrate/deposit assembly is pressed on the paper at a pressure of 2.5 kg for 30 seconds. Following removal of the substrate/deposit assembly, some of the deposit has transferred to the paper. The colour of the deposit transfer to the paper is then measured using a Minolta CR300 colorimeter, the colour being characterized by the colorimetric parameters L*, a*, b*. The colorimetric parameters L*₀, a*₀, b*₀ of the colour of the plain paper used are determined.

A determination is then made of the colour difference ΔE1 between the colour of the deposit transferred relative to the colour of the plain paper, using the following relationship:

ΔE1=\sqrt{(L*−L₀*)²+(a*−a₀*)²+(b*−b₀*)²}

Separately, a total transfer reference is prepared by applying the mixture directly to a piece of paper identical to that used above, at ambient temperature (25° C.), by spreading the composition by means of a fine brush to give a deposit of the composition of approximately 15 μm, which is then left to dry for 30 minutes at ambient temperature (25° C.). After drying, a direct measurement is made of the colorimetric parameters L*′, a*′, b*′ of the colour of the deposit placed onto the paper, corresponding to the total transfer reference colour. The colorimetric parameters L*′₀, a*′₀, b*′₀ of the colour of the plain paper used are determined.

A determination is then made of the colour difference ΔE2 between the total transfer reference colour relative to the colour of the plain paper, using the following relationship:

ΔE2=\sqrt{(L*′−L₀*′)²+(a*′−a₀*′)²+(b*′−b₀*′)²}

The transfer of the composition, expressed as a percentage, is equal to the following ratio:

100×ΔE1/ΔE2

The measurement is carried out on 4 substrates in succession, and the transfer value corresponds to the average of the 4 measurements obtained with the 4 substrates.

### Hardness of the Deposit

The hardness of the film obtained is measured
either following deposition on a glass plate of a layer 300 μm thick (before drying) of the composition containing the compounds X and Y and the compatible oil
or by successive depositions on a glass plate of the two compositions containing, respectively, the compounds X and Y, one at least of the compositions containing the compatible oil, such that the final deposit has a thickness before drying of 300 μm.

The glass plate is dried on a support and held at 30° C. for 23 hours in an ambient atmosphere and then for 1 hour at 70% relative humidity.

The hardness of the film obtained is then measured in accordance with the standard ASTM D-43-66 or the standard NF-T 30-016 (December 1991), using a Persoz pendulum.

Advantageously the film obtained containing the reaction product of X and Y and the compatible oil has a Persoz hardness of less than or equal to 60 s, preferably between 30 and 50 seconds, preferably between 15 and 30 s.

### Other Oils

At least one of the compositions advantageously comprises, in addition to the aforementioned oils, an oil selected from non-volatile oils.

In one embodiment at least one of the first and second compositions used according to the invention comprises at least one non-volatile oil which more particularly is selected from non-volatile hydrocarbon oils and/or fluoro oils.

As non-volatile hydrocarbon oil, mention may in particular be made of:
hydrocarbon oils of plant origin, such as triesters of fatty acids and of glycerol, the fatty acids of which can have varied chain lengths from C₄ to C₂₄, it being possible for the latter to be linear or branched and saturated or unsaturated; these oils are in particular wheatgerm, sunflower, grape seed, sesame, maize, apricot, castor, shea, avocado, olive, soybean, sweet almond, palm, rapeseed, cotton seed, hazelnut, macadamia, jojoba, alfalfa, poppy, potimarron, sesame, marrow, blackcurrant, evening primrose, millet, barley, quinoa, rye, safflower, candlenut, passion flower or musk rose oil; or else triglycerides of caprylic/capric acids, such as those sold by the company Stéarineries Dubois or those sold under the names Miglyol 810, 812 and 818 by the company Dynamit Nobel,
synthetic ethers having from 10 to 40 carbon atoms;
apolar hydrocarbon oils such as squalene, linear or branched hydrocarbons such as paraffin oils, petrolatum oils and naphthalene oils, hydrogenated or partially hydrogenated polyisobutene, isoeicosane, squalene, decene/butene copolymers, polybutene/polyisobutene copolymers, more particularly Indopol L-14, polydecenes such as Puresyn 10, and mixtures thereof;
liquid fatty alcohols at ambient temperature with a branched and/or unsaturated carbon chain having from 12 to 26 carbon atoms such as octyldodecanol, isostearylic alcohol, oleic alcohol, 2-hexyldecanol, 2-butyloctanol, 2-undecylpentadecanol;
higher fatty acids, such as oleic acid, linoleic acid or linolenic acid;
carbonates,
acetates,
citrates,
and mixtures thereof.

The non-volatile oil may be present in an amount of from 0.1% to 80% by weight, preferably from 1% to 60% by weight, more preferably from 5% to 50% by weight and more preferably still from 14% to 40% by weight relative to the total weight of each first and second composition or relative to the total weight of the composition when X and Y are present within the same composition.

In one embodiment the first and second compositions employed in the method according to the invention are anhydrous.

### Aqueous Phase

At least one composition may comprise an aqueous phase.

The aqueous phase may be composed essentially of water; it may also comprise a mixture of water and water-miscible solvent (with a miscibility in water of more than 50% by weight at 25° C.), such as lower monoalcohols having 1 to 5 carbon atoms, such as ethanol and isopropanol, glycols having 2 to 8 carbon atoms such as propylene glycol, ethylene glycol, 1,3-butylene glycol and dipropylene glycol, C₃-C₄ ketones, C₂-C₄ aldehydes and mixtures thereof.

The aqueous phase (water and, where appropriate, the water-miscible solvent) may be present in an amount of from 5% to 95% by weight, relative to the total weight of each composition, preferably from 10% to 85% by weight and more preferably from 2% to 80% by weight.

### Solid Fatty Substances

At least one of the compositions of the kit may further comprise at least one fatty substance which is solid at ambient temperature (25° C.), preferably having a melting point of more than 25° C., selected more particularly from waxes, pasty lipids and mixtures thereof. These fatty substances may be animal, plant, mineral or synthetic in origin.

### Wax

“Wax” is understood to mean a lipophilic compound which is solid at ambient temperature (25° C.) and exhibits a reversible solid/liquid state change, having a melting point greater than or equal to 30° C. and possibly up to 120° C.

In particular the waxes suitable for the invention may have a melting point greater than approximately 45°, and in particular greater than 55° C.

The melting point of the wax can be measured using a differential scanning calorimeter (DSC), an example being the calorimeter sold under the name DSC 30 by Mettler.

Use may be made in particular of hydrocarbon waxes such as beeswax, lanolin wax and Chinese insect waxes; rice wax, carnauba wax, candelilla wax, ouricury wax, alfa wax, cork fibre wax, sugarcane wax, Japan wax and sumac wax; montan wax, microcrystalline waxes, paraffins and ozokerite; polyethylene waxes, waxes obtained by Fischer-Tropsch synthesis, and waxy copolymers, and also their esters.

Mention may also be made of the waxes obtained by catalytic hydrogenation of animal or plant oils having C8-C32 linear or branched fatty chains.

Among these, mention may be made more particularly of hydrogenated jojoba oil, isomerized jojoba oil, such as the trans isomerized partially hydrogenated jojoba oil manufactured and sold by Desert Whale under the commercial reference ISO-JOJOBA-50®, hydrogenated sunflower oil, hydrogenated castor oil, hydrogenated copra oil and hydrogenated lanolin oil, the di(1,1,1-trimethylolpropane) tetrastearate sold under the name Hest 2T-4S by Heterene and the di(1,1,1-trimethylolpropane) tetrabehenate sold under the name Hest 2T-4B by Heterene.

Mention may also be made of silicone waxes such as alkyl- or alkoxydimethicones having 16 to 45 carbon atoms, and fluoro waxes.

It is also possible to use the wax obtained by hydrogenating olive oil esterified with stearyl alcohol, which is sold under the name Phytowax Olive 18 L 57 or else the waxes obtained by hydrogenating castor oil esterified with cetyl alcohol, which is sold under the name Phytowax ricin 16L64 and 22L73, by Sophim. Waxes of this kind are described in Patent Application FR-A-2792190.

### Pasty Compounds

The composition according to the invention may further comprise at least one pasty compound. By “pasty” in the sense of the present invention is meant a lipophilic fatty compound featuring a reversible solid/liquid state change and exhibiting in the solid state an anisotropic crystalline organization, and at a temperature of 23° C. comprising a liquid fraction and a solid fraction. The term “pasty compound” also refers to polyvinyl laurate.

The pasty compound is preferably selected from synthetic compounds and compounds of plant origin. A pasty compound is obtainable by synthesis from starting products of plant origin.

The pasty compound is advantageously selected from
lanolin and its derivatives
polyol ethers selected from the ethers of pentaerythritol and of polyalkylene glycol, ethers of fatty alcohol and of sugar, and mixtures thereof: the pentaerythritol and polyethylene glycol ether containing 5 oxyethylene units (5 OE) (CTFA name: PEG-5 Pentaerythrityl Ether), the pentaerythritol and polypropylene glycol ether containing 5 oxypropylene units (5 OP) (CTFA name: PPG-5 Pentaerythrityl Ether), and their mixtures, and more specifically the PEG-5 Pentaerythrityl Ether, PPG-5 Pentaerythrityl Ether and soya oil mixture sold under the name Lanolide by the company Vevy, in which the constituents are in a 46/46/8 weight ratio: 46% of PEG-5 Pentaerythrityl Ether, 46% of PPG-5 Pentaerythrityl Ether and 8% of soya oil
polymeric or non-polymeric silicone compounds
polymeric or non-polymeric fluorine compounds
vinyl polymers, more particularly
   olefin homopolymers
   olefin copolymers
   hydrogenated diene homopolymers and copolymers
   linear or branched oligomers, homopolymers or copolymers of alkyl (meth)acrylates preferably having a C₈-C₃₀ alkyl group
   oligomers, homopolymers and copolymers of vinyl esters having C₈-C₃₀ alkyl groups
   oligomers, homopolymers and copolymers of vinyl ethers having C₈-C₃₀ alkyl groups,
fat-soluble polyethers resulting from poly-etherification between one or more C2-C100, preferably C2-C50, diols,
esters,
   and mixtures thereof.

A preferred fluorine-containing pasty silicone compound is the polymethyltrifluoropropylmethylalkyldimethylsiloxane manufactured under the name X22-1088 by Shin Etsu.

Among the fat-soluble polyethers, preference is given in particular to the copolymers of ethylene oxide and/or of propylene oxide with long-chain C6-C30 alkylene oxides, more preferably such that the weight ratio of the ethylene oxide and/or propylene oxide with alkylene oxides in the copolymer is from 5:95 to 70:30. In this class, mention may be made more particularly of the copolymers such that the long-chain alkylene oxides are arranged in blocks with an average molecular weight of 1000 to 10 000, for example a polyoxyethylene/polydodecyl glycol block copolymer such as the ethers of dodecanediol (22 mol) and of polyethylene glycol (45 EO) that are sold under the brand name Elfacos ST9 by Akzo Nobel.

Among the esters, preference is given more particularly to:
the esters of an oligomeric glycerol, more particularly the esters of diglycerol, in particular the condensates of adipic acid and glycerol for which some of the hydroxyl groups of the glycerols have reacted with a mixture of fatty acids such as stearic acid, capric acid, stearic acid and isostearic acid and 12-hydroxystearic acid, of the kind, more particularly, sold under the brand name Softisan 649 by the company Sasol,
the arachidyl propionate sold under the brand name Waxenol 801 by Alzo,
phytosterol esters,
fatty acid triglycerides and their derivatives,
pentaerythritol esters,
noncrosslinked polyesters resulting from the polycondensation of a linear or branched C4-C50 dicarboxylic or polycarboxylic acid and a C2-C50 diol or polyol,
aliphatic esters of an ester resulting from the esterification of an aliphatic hydroxycarboxylic ester by an aliphatic carboxylic acid.

The aliphatic carboxylic acid contains 4 to 30 and preferably 8 to 30 carbon atoms. It is preferably selected from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid, docosanoic acid, and mixtures thereof.

The aliphatic carboxylic acid is preferably branched.

Aliphatic hydroxycarboxylic ester is advantageously obtained from a hydroxyl-containing aliphatic carboxylic acid containing 2 to 40 carbon atoms, preferably 10 to carbon atoms and more preferably 12 to 28 carbon atoms and 1 to 20 hydroxyl groups, preferably 1 to 10 hydroxyl groups and more preferably 1 to 6 hydroxyl groups. The aliphatic hydroxycarboxylic ester is selected from:

a) the partial or total esters of saturated linear monohydroxylated aliphatic monocarboxylic acids;
b) the partial or total esters of unsaturated monohydroxylated aliphatic monocarboxylic acids;
c) the partial or total esters of saturated monohydroxylated aliphatic polycarboxylic acids;
d) the partial or total esters of saturated polyhydroxylated aliphatic polycarboxylic acids;
e) the partial or total esters of C₂ to C₁₆ aliphatic polyols which have reacted with a mono- or polyhydroxylated aliphatic mono- or polycarboxylic acid, and mixtures thereof.

The aliphatic esters of an ester are advantageously selected from:
the ester resulting from the esterification reaction of hydrogenated castor oil with isostearic acid in 1 to 1 (1/1) proportions or hydrogenated castor oil monoisostearate,
the ester resulting from the esterification reaction of hydrogenated castor oil with isostearic acid in 1 to 2 proportions (1/2) or hydrogenated castor oil diisostearate,
the ester resulting from the esterification reaction of hydrogenated castor oil with isostearic acid in 1 to 3 proportions (1/3) or hydrogenated castor oil triisostearate,
and mixtures thereof.

The nature and the amount of the solid fatty substances are a function of the desired mechanical properties and textures. As an indication, the waxes may represent from 0.1% to 70% by weight, relative to the total weight of each composition, more preferably from 1% to 40% and more preferably still from 5% to 30% by weight.

### Film-Former

The compositions may comprise a film-forming polymer.

According to one embodiment, the composition contains at least one polymer that may be chosen from film-forming polymers. Certain film-forming polymers may be gelling agents. According to one embodiment, the polymer is not a nitrocellulose.

For the purposes of the invention, the term “polymer” means a compound containing at least 2 repeating units and preferably at least 3 repeating units.

The term “film-forming polymer” means a polymer capable, by itself or in the presence of an auxiliary film-forming agent, of forming a macroscopically continuous film on a support, especially on keratin materials, preferably a cohesive film and better still a film whose cohesion and mechanical properties are such that said film can be isolated from said support.

The polymer may be present in the composition in an amount ranging from 0.1% to 60% by weight, relative to the total weight of the composition, preferably ranging from 0.1% to 50% by weight, preferably ranging from 0.5% to 40% by weight, preferentially ranging from 1% to 30% by weight and more preferentially ranging from 1% to 25% by weight.

In one embodiment, the film-forming organic polymer is at least one polymer chosen from the group comprising:
film-forming polymers that are soluble in an organic liquid medium, in particular liposoluble polymers, when the organic liquid medium comprises at least one oil;
film-forming polymers that are dispersible in an organic solvent medium, in particular polymers in the form of non-aqueous dispersions of polymer particles, preferably dispersions in silicone oils or hydrocarbon-based oils; in one embodiment, the non-aqueous polymer dispersions comprise polymer particles that are surface-stabilized with at least one stabilizer;
film-forming polymers in the form of aqueous dispersions of polymer particles, often known as “latices”; in this case, the composition comprises an aqueous phase;
water-soluble film-forming polymers; in this case, the composition comprises an aqueous phase.

Among the film-forming polymers that may be used in the composition of the present invention, mention may be made of synthetic polymers, of free-radical type or of polycondensate type, polymers of natural origin, and mixtures thereof. Film-forming polymers that may be mentioned in particular include acrylic polymers, polyurethanes, polyesters, polyamides, polyureas, cellulosic polymers, for instance nitrocellulose, silicone polymers, in particular silicone resins, silicone-grafted acrylic polymers, polyamide polymers and copolymers, and polyisoprenes.

The film-forming polymer may be chosen from the film-forming polymers described in patent application WO 04/028487, the content of which is incorporated into the present patent application by way of reference.

In particular, the film-forming polymer may be a cross-linked silicone compound as described in patent applications US 2003/0 103 918 and US 2003/0 049 216, the content of which is incorporated into the present patent application by way of reference.

Film-forming polymers are especially described in the international patent application filed under No. PCT/FR03/02849, the content of which is incorporated by way of reference.

The film-forming polymer may be a film-forming linear ethylenic block polymer, which is advantageously styrene-free. More preferably, the block polymer comprises at least one first block and at least one second block that have different glass transition temperatures (Tg), said first and second blocks being linked together via an intermediate block comprising at least one constituent monomer of the first block and at least one constituent monomer of the second block.

The film-forming polymer may also be in the form of a dispersion of particles, which are preferably solid, of a grafted ethylenic polymer in a liquid fatty phase. Such a dispersion is especially described in the international patent application filed under No. PCT/FR03/03709, the content of which is incorporated by way of reference.

Advantageously, the grafted ethylenic polymer comprises an ethylenic skeleton that is insoluble in said liquid fatty phase, and side chains that are covalently bonded to said skeleton and are soluble in said dispersion medium.

### Amphiphilic Silicone

The compositions may comprise at least one amphiphilic silicone.

The amphiphilic silicones which can be used contain a silicone portion which is compatible with a silicone medium, and a hydrophilic portion which may be, for example, the residue of a compound selected from alcohols and polyols, having 1 to 12 hydroxyl groups, polyoxyalkylenes containing at least two oxyalkylene units and having from 0 to 20 oxypropylene units and/or from 0 to 20 oxyethylene units. This hydrophilic portion therefore exhibits affinity for the hydrophilic particles and contributes to their dispersion in a silicone medium.

The amphiphilic silicone may be an oil without gelling activity. Oils of this kind may be composed:
of dimethicone copolyols optionally containing phenyl groups,
of alkylmethicone copolyols,
of polyglycerol silicones, in other words silicones having alkylglyceryl ether groups,
of silicones having perfluorinated side groups and having glycerol side groups,
of silicones having polyoxyethylene/polyoxypropylene side groups and having perfluorinated side groups,
of block copolymers of silicone and hydrophilic block copolymers other than the polyether, for example polyoxazoline or polyethyleneimine,
of graft copolymers of silicone-grafted polysaccharide type,
of silicone block, poly(ethylene oxide/propylene oxide) block copolymers.

The amphiphilic silicone that can be used may also be an at least partly crosslinked amphiphilic silicone resin.

Possible examples of such resins include the following:
crosslinked silicone resins having alkylpolyether groups, such as the polyethylene oxide (PEO) and the polyethylene oxide/polypropylene oxide (PEO/PPO) that are described in U.S. Pat. No. 5,412,004, and
silicone resins partly crosslinked by α,ω-dienes, possessing hydrophilic PEO/PPO side chains and also hydrophobic alkyl side chains, such as those described in EP-A-1 048 686. The hydrophilic side chains are obtained by a reaction with a PEO/PPO at a single vinyl end, and the alkyl side chains are formed by a reaction with an α-olefin having a fatty chain.

In the amphiphilic silicone resin the silicone portion is composed advantageously of polydimethylsiloxane.

### Polyamide Polymer or Copolymer

The compositions may comprise at least one polyamide polymer or copolymer which may be chosen from polyamide homopolymers, polyamides branched with fatty chains, polyamide-organosiloxanes, polyamide-polyester copolymers and polyamide-polyacrylic copolymers, and mixtures thereof.

As polyamide polymers that may be used in the invention, mention may be made of the polymers described in patent application EP 1 343 458, the content of which is incorporated into the present patent application by reference, for example the polyamide resins resulting from the condensation of an aliphatic dicarboxylic acid and a diamine (including compounds containing more than 2 carbonyl groups and 2 amine groups), the carbonyl and amine groups of adjacent individual units being condensed via an amide bond. These polyamide resins are especially the products sold under the brand name Versamid® by the companies General Mills, Inc. and Henkel Corp. (Versamid 930, 744 or 1655) or by the company Olin Mathieson Chemical Corp., under the brand name Onamid®, especially Onamid S or C. These resins have a weight-average molecular mass ranging from 6000 to 9000. For further information regarding these polyamides, reference may be made to documents U.S. Pat. No. 3,645,705 and U.S. Pat. No. 3,148,125. Versamid® 930 or 744 is more especially used.

It is also possible to use the polyamides sold by the company Arizona Chemical under the references Uni-Rez (2658, 2931, 2970, 2621, 2613, 2624, 2665, 1554, 2623 and 2662) and the product sold under the reference Macromelt 6212 by the company Henkel. For further information regarding these polyamides, reference may be made to the document U.S. Pat. No. 5,500,209.

It is also possible to use vegetable-based polyamide resins, for instance those described in U.S. Pat. No. 5,783,657 and U.S. Pat. No. 5,998,570.

As polyamide polymers that may be used in the invention, mention may be made of the polyamides branched with pendent fatty chains and/or terminal fatty chains containing from 6 to 120 carbon atoms, better still 8 to 120 and especially from 12 to 68 carbon atoms, each terminal fatty chain being linked to the polyamide skeleton via at least one linking group, in particular ester. Preferably, these polymers comprise a fatty chain at each end of the polymer skeleton and in particular of the polyamide skeleton. Other linking groups that may be mentioned include ether, amine, urea, urethane, thioester, thiourea and thiourethane groups.

These polymers are more especially those described in document U.S. Pat. No. 5,783,657 from the company Union Camp. Examples that may be mentioned include the commercial products sold by the company Arizona Chemical under the names Uniclear 80 and Uniclear 100. They are sold, respectively, in the form of a gel at 80% (of active material) in a mineral oil, and at 100% (of active material). They have a softening point of from 88 to 94° C. These commercial products are a mixture of copolymers of a C₃₆ diacid condensed with ethylenediamine, with a weight-average molecular mass of about 6000. The ester end groups result from the esterification of the remaining acid end groups with cetyl alcohol, stearyl alcohol or mixtures thereof (also known as cetyl stearyl alcohol).

As polyamide polymers that may be used in the invention, mention may also be made of polyamides comprising at least one polyorganosiloxane group, consisting of from 1 to 1000 organosiloxane units in the main chain or in the form of a graft. The polymers are, for example, those described in documents U.S. Pat. No. 5,874,069, U.S. Pat. No. 5,919,444, U.S. Pat. No. 6,051,216, U.S. Pat. No. 5,981,680 and WO 04/054 524, the content of which is incorporated into the present patent application by way of reference.

The silicone polyamide may be a polymer comprising at least one unit of formula (IV) or (V):

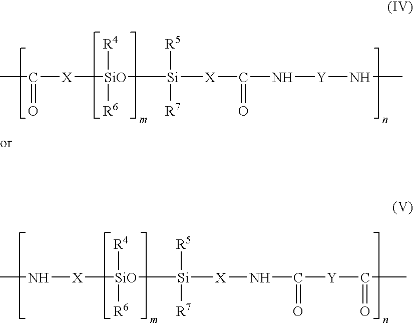

R⁴, R⁵, R⁶ and R⁷, which are identical or different, represent a group chosen from:
saturated or unsaturated, C₁ to C₄₀ linear, branched or cyclic hydrocarbon groups, which may contain in their chain one or more oxygen, sulphur and/or nitrogen atoms, and which may be partially or totally substituted by fluorine atoms,
C₆ to C₁₀ aryl groups, optionally substituted by one or more C₁ to C₄ alkyl groups,
polyorganosiloxane chains possibly containing one or more oxygen, sulphur and/or nitrogen atoms,
X, which are identical or different, represent a linear or branched C₁ to C₃₀ alkylenediyl group, which may contain in its chain one or more oxygen and/or nitrogen atoms,
Y is a C₁ to C₅₀ saturated or unsaturated, linear or branched alkylene, arylene, cycloalkylene, alkylarylene or arylalkylene divalent group, which may comprise one or more oxygen, sulphur and/or nitrogen atoms, and/or which may bear as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ aminoalkyl groups, or
n is an integer ranging from 2 to 500 and preferably from 2 to 200, and m is an integer ranging from 1 to 1000, preferably from 1 to 700 and better still from 6 to 200.

### Surfactants

The compositions according to the invention may contain emulsifying surfactants, which are especially present in a proportion ranging from 0.1% to 30% by weight, better still from 1% to 15% and even better still from 2% to 10% relative to the total weight of each composition. These surfactants may be chosen from anionic, nonionic, amphoteric and zwitterionic surfactants. Reference may be made to the document “Encyclopedia of Chemical Technology, Kirk-Othmer”, Volume 22, pp. 333-432, 3rd Edition, 1979, Wiley, for the definition of the properties and functions (emulsifying) of surfactants, in particular pp. 347-377 of this reference, for the anionic and nonionic surfactants.

The surfactants preferentially used in the first and second compositions according to the invention are chosen from:

a) nonionic surfactants with an HLB of greater than or equal to 8 at 25° C., used alone or as a mixture; mention may be made especially of:
oxyethylenated and/or oxypropylenated ethers (which may comprise from 1 to 150 oxyethylene and/or oxypropylene groups) of glycerol;
oxyethylenated and/or oxypropylenated ethers (which may comprise from 1 to 150 oxyethylene and/or oxypropylene groups) of fatty alcohols (especially of C₈-C₂₄ and preferably C₁₂-C₁₈ alcohol), such as oxyethylenated cetearyl alcohol ether containing 30 oxyethylene groups (CTFA name Ceteareth-30) and the oxyethylenated ether of the mixture of C₁₂-C₁₅ fatty alcohols comprising 7 oxyethylene groups (CTFA name C12-15 Pareth-7 sold under the name Neodol 25-7® by Shell Chemicals);
fatty acid esters (especially of a C₈-C₂₄ and preferably C₁₆-C₂₂ acid) of polyethylene glycol (which may comprise from 1 to 150 ethylene glycol units), such as PEG-50 stearate and PEG-40 monostearate sold under the name Myrj 52P by the company ICI Uniqema;
fatty acid esters (especially of a C₈-C₂₄ and preferably C₁₆-C₂₂ acid) of oxyethylenated and/or oxypropylenated glyceryl ethers (which may comprise from 1 to 150 oxyethylene and/or oxypropylene groups), for instance PEG-200 glyceryl monostearate sold under the name Simulsol 220™ by the company SEPPIC; glyceryl stearate polyethoxylated with 30 ethylene oxide groups, for instance the product Tagat S sold by the company Goldschmidt, glyceryl oleate polyethoxylated with 30 ethylene oxide groups, for instance the product Tagat O sold by the company Goldschmidt, glyceryl cocoate polyethoxylated with 30 ethylene oxide groups, for instance the product Varionic LI 13 sold by the company Sherex, glyceryl isostearate polyethoxylated with 30 ethylene oxide groups, for instance the product Tagat L sold by the company Goldschmidt, and glyceryl laurate polyethoxylated with 30 ethylene oxide groups, for instance the product Tagat I from the company Goldschmidt;
fatty acid esters (especially of a C₈-C₂₄ and preferably C₁₆-C₂₂ acid) of oxyethylenated and/or oxypropylenated sorbitol ethers (which may comprise from 1 to 150 oxyethylene and/or oxypropylene groups), for instance polysorbate 60 sold under the name Tween 60 by the company Uniqema;
dimethicone copolyol, such as the product sold under the name Q2-5220 by the company Dow Corning;
dimethicone copolyol benzoate (Finsolv SLB 101 and 201 by the company Fintex);
copolymers of propylene oxide and of ethylene oxide, also known as EO/PO polycondensates, for instance the polyethylene glycol/polypropylene glycol/polyethylene glycol triblock polycondensates sold under the name Synperonic, for instance Synperonic PE/L44 and Synperonic PE/F127, by the company ICI, and mixtures thereof;
and mixtures thereof.
   b) nonionic surfactants with an HLB of less than 8 at 25° C., optionally combined with one or more nonionic surfactants with an HLB of greater than 8 at 25° C., as mentioned above, such as:
saccharide esters and ethers, such as sucrose stearate, sucrose cocoate and sorbitan stearate, and mixtures thereof, for instance Arlatone 2121 sold by the company ICI;
fatty acid esters (especially of a C₈-C₂₄ and preferably C₁₆-C₂₂ acid) of polyols, especially of glycerol or of sorbitol, such as glyceryl stearate, glyceryl stearate such as the product sold under the name Tegin M by the company Goldschmidt, glyceryl laurate such as the product sold under the name Imwitor 312 by the company Hills, polyglyceryl-2 stearate, sorbitan tristearate or glyceryl ricinoleate;
the mixture of cyclomethicone/dimethicone copolyol sold under the name Q2-3225C by the company Dow Corning.
   c) anionic surfactants such as:
C₁₆-C₃₀ fatty acid salts, especially those derived from amines, for instance triethanolamine stearate;
polyoxyethylenated fatty acid salts, especially those derived from amines or alkali metal salts, and mixtures thereof;
phosphoric esters and salts thereof, such as DEA oleth-10 phosphate (Crodafos N 10N from the company Croda) and cetyl phosphate (Amphisol K from the company DSM Nutritional Products);
sulphosuccinates such as Disodium PEG-5 citrate lauryl sulphosuccinate and Disodium ricinoleamido MEA sulphosuccinate;
alkyl ether sulphates, such as sodium lauryl ether sulphate;
isethionates;
acylglutamates such as Disodium hydrogenated tallow glutamate (Amisoft HS-21 R sold by the company Ajinomoto), and mixtures thereof.

Triethanolamine stearate is most particularly suitable for the invention. This is generally obtained by simple mixing of stearic acid and triethanolamine.

### Gelling Agent

The compositions may comprise at least one gelling agent, which is an agent comprising at least one organic compound.

An aqueous-medium gelling agent may be selected from:
water-soluble cellulosic thickeners,
guar gum, xanthan gum, carob gum, scleroglucan gum, gellan gum, rhamsam gum, karaya gum and carrageenan gum,
alginates, maltodextrins, starch and its derivatives, hyaluronic acid and its salts,
chitosans and their derivatives,
polyglyceryl (meth)acrylate polymers sold under the names “Hispagel” or “Lubragel” by the Hispano Quimica or Guardian companies,
polyvinylpyrrolidone,
polyvinyl alcohol,
crosslinked acrylamide polymers and copolymers, such as those sold under the names “PAS 5161” or “Bozepol C” by the Hoechst company or “Sepigel 305” by the Seppic company by the Allied Colloid company, or else
crosslinked methacryloyloxyethyltrimethylammonium chloride homopolymers sold under the name “Salcare SC95” by the Allied Colloid company,
associative polymers and more particularly associative polyurethanes,
polyamides such as Uniclear 100 sold by Arizona,
the above-described semi-crystalline polymers,
organosiloxanes obtained by hydroxysilylation.

Gelling agents of this kind are more particularly described in patent application EP-A-1400234, whose content is incorporated by way of reference.

An oily-medium gelling agent may be selected from
alkylated guar gums (with a C₁-C₆ alkyl group) such as those described in EP-A-708114;
oil-gelling polymers such as the triblock or star polymers resulting from the polymerization or copolymerization of at least one monomer having an ethylene group, such as the polymers sold under the name Kraton;
polymers with a weight-average molecular mass of less than 100 000, comprising a) a polymeric skeleton having repeating hydrocarbon units including at least one heteroatom, and optionally b) at least one pendent fatty chain and/or at least one terminal fatty chain, these chains being optionally functionalized, having 6 to 120 carbon atoms and being bonded to these hydrocarbon units, as are described in patent applications WO-A-02/056847 and WO-A-02/47619, whose content is incorporated by way of reference; in particular, polyamide resins (more particularly those including alkyl groups having 12 to 22 carbon atoms) such as those described in U.S. Pat. No. 5,783,657, whose content is incorporated by way of reference.

The organic gelling agents may be selected from those described in patent application WO-A-03/105788, whose content is incorporated by way of reference.

As a gelling agent which can be used in the invention mention may also be made of the crosslinked organopolysiloxanes obtained by hydroxysilylation. Possible examples of resins obtained by hydroxysilylation that can be used in accordance with the invention include those sold under the names KSG6 from Shin-Etsu, Trefil E-505C or Trefil E-506C from Dow-Corning, Gransil from Grant Industries (SR-CYC, SR DMF10, SR-DC556), or those sold in the form of preconstituted gels (KSG15, KSG17, KSG16, KSG18, KSG20, KSG21, KSG31, KSG32 from Shin-Etsu, Gransil SR 5CYC gel, Gransil SR DMF 10 gel, Gransil SR DC556 gel, SF 1204 and JK 113 from General

Electric). The Dow Corning products DC9010 and DC 9011 and Wacker product EF199913 may also be used; these products all comprise oxyethylene groups, just like the products KSG20, KSG21, KSG31 and KSG32. A mixture of these commercial products can also be used. These products are obtained in particular as described in U.S. Pat. No. 5,236,986 of Shin-Etsu, U.S. Pat. No. 5,412,004 of Kosé and Shin-Etsu or else U.S. Pat. No. 5,811,487 of Dow Corning.

### Colorant

The compositions may comprise at least one colorant chosen, for example, from pigments, nacres, dyes and materials with an effect, and mixtures thereof.

These colorants may be present in a content ranging from 0.01% to 50% by weight and preferably from 0.01% to 30% by weight relative to the weight of each first and second composition or relative to the total weight of the composition when X and Y are present in the same composition.

The pigments that are useful in the present invention may be in the form of powder or of pigmentary paste.

The term “dyes” should be understood as meaning compounds, generally organic, which are soluble in at least one oil or in an aqueous-alcoholic phase.

The term “pigments” should be understood as meaning white or coloured, mineral or organic particles, which are insoluble in an aqueous medium, and which are intended to colour and/or opacify the resulting film.

The term “nacres” or nacreous pigments should be understood as meaning coloured particles of any form, which may or may not be iridescent, especially produced by certain molluscs in their shell or else synthesized, and which have a colour effect via optical interference.

The pigment may be an organic pigment. The term “organic pigment” means any pigment that satisfies the definition in Ullmann's encyclopaedia in the chapter on organic pigments. The organic pigment may especially be chosen from nitroso, nitro, azo, xanthene, quinoline, anthraquinone, phthalocyanine, metal complex, isoindolinone, isoindoline, quinacridone, perinone, perylene, diketopyrrolopyrrole, thioindigo, dioxazine, triphenylmethane and quinophthalone compounds.

The organic pigment(s) may be chosen, for example, from carmine, carbon black, aniline black, melanin, azo yellow, quinacridone, phthalocyanin blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenolic derivatives as described in patent FR 2 679 771.

These pigments may also be in the form of composite pigments as described in patent EP 1 184 426. These composite pigments may be composed especially of particles comprising an inorganic nucleus at least partially coated with an organic pigment and at least one binder to fix the organic pigments to the nucleus. Examples that may also be mentioned include pigmentary pastes of organic pigments such as the products sold by the company Hoechst under the names:
Jaune Cosmenyl IOG: Pigment Yellow 3 (CI 11710);
Jaune Cosmenyl G: Pigment Yellow 1 (CI 11680);
Orange Cosmenyl GR: Pigment Orange 43 (CI 71105);
Rouge Cosmenyl R″: Pigment Red 4 (CI 12085);
Carmine Cosmenyl FB: Pigment Red 5 (CI 12490);
Violet Cosmenyl RL: Pigment Violet 23 (CI 51319);
Bleu Cosmenyl A2R: Pigment Blue 15.1 (CI 74160);
Vert Cosmenyl GG: Pigment Green 7 (CI 74260);
Noir Cosmenyl R: Pigment Black 7 (CI 77266).

The pigment may also be a lake. The term “lake” means insolubilized dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate, and aluminium.

Among the organic dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 10 (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a pigment with special effects. The term “pigments with special effects” means pigments that generally create a non-uniform coloured appearance (characterized by a certain shade, a certain vivacity and a certain lightness) that changes as a function of the conditions of observation (light, temperature, observation angles, etc.). They thus contrast with white or coloured pigments that afford a standard uniform opaque, semi-transparent or transparent shade. Two types of pigment with special effects exist: those with a low refractive index, such as fluorescent, photochromic or thermochromic pigments, and those with a high refractive index, such as nacres or flakes.

Pigments with special effects that may be mentioned include nacreous pigments such as white nacreous pigments such as mica coated with titanium or with bismuth oxychloride, coloured nacreous pigments such as titanium mica with iron oxides, titanium mica especially with ferric blue or with chromium oxide, titanium mica with an organic pigment of the abovementioned type, and also nacreous pigments based on bismuth oxychloride.

Mention may also be made of pigments with an interference effect that are not fixed onto a substrate, for instance liquid crystals (Helicones HC from Wacker), holographic interference flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

Quantum dots are luminescent semiconductive nanoparticles capable of emitting, under light excitation, irradiation with a wavelength of between 400 nm and 700 nm. These nanoparticles are known from the literature. They may be manufactured in particular according to the processes described, for example, in U.S. Pat. No. 6,225,198 or U.S. Pat. No. 5,990,479, in the publications cited therein, and also in the following publications: Dabboussi B. O. et al. “(CdSe)ZnS core-shell quantum dots: synthesis and characterization of a size series of highly luminescent nanocrystallites” Journal of Physical Chemistry B, vol. 101, 1997, pp. 9463-9475 and Peng, Xiaogang et al. “Epitaxial growth of highly luminescent CdSe/CdS core/shell nanocrystals with photostability and electronic accessibility”, Journal of the American Chemical Society, vol. 119, No. 30, pp. 7019-7029.

Pigments with special effects also comprise fluorescent pigments, whether these are substances that are fluorescent in daylight or that produce an ultraviolet fluorescence, phosphorescent pigments, photochromic pigments and thermochromic pigments.

The pigment may be a mineral pigment. The term “mineral pigment” means any pigment that satisfies the definition in Ullmann's encyclopaedia in the chapter on inorganic pigments. Among the mineral pigments that are useful in the present invention, mention may be made of zirconium oxide or cerium oxide, and also iron oxide or chromium oxide, manganese violet, ultramarine blue, chromium hydrate, ferric blue and titanium dioxide. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

The pigment may also be a nacreous pigment such as white nacreous pigments, for example mica coated with titanium or with bismuth oxychloride, coloured nacreous pigments such as mica coated with titanium and with iron oxides, mica coated with titanium and especially with ferric blue or chromium oxide, mica coated with titanium and with an organic pigment as defined above, and also nacreous pigments based on bismuth oxychloride. Examples that may be mentioned include the Cellini pigments sold by Engelhard (Mica-TiO₂-lake), Prestige sold by Eckart (Mica-TiO₂) or Colorona sold by Merck (Mica-TiO₂—Fe₂O₃).

In addition to nacres on a mica support, multilayer pigments based on synthetic substrates such as alumina, silica, calcium sodium borosilicate or calcium aluminium borosilicates, and aluminium, may be envisaged.

The size of the pigment that is useful in the context of the present invention is generally between 10 nm and 200 μm, preferably between 20 nm and 80 μm and more preferentially between 30 nm and 50 μm.

According to one preferred embodiment the composition according to the invention may comprise fillers.

The term “fillers” should be understood to mean colourless or white, mineral or synthetic particles of any shape, which are insoluble in the medium of the composition regardless of the temperature at which the composition is manufactured.

The fillers may be platelet, spherical or oblong inorganic or organic fillers of any shape, regardless of the crystallographic shape (for example sheet, cubic, hexagonal, orthorhombic, and the like). Mention may be made of talc, mica, silica, kaolin, polyamide powders (Nylon®), poly-β-alanine powders, polyethylene powders, polymethyl methacrylates, polyurethane powders such as the hexamethylene diisocyanate and trimethylol hexyl lactone copolymer powder sold under the names Plastic Powder D-400 by the company Toshiki, tetrafluoroethylene polymer powders (Teflon®), micronized wax particles, in particular carnauba microwaxes such as those marketed under the name “MicroCare 350®” by the company Micro Powders, synthetic wax microwaxes such as those marketed under the name “MicroEase 114S®” by the company Micro Powders, microwaxes consisting of a mixture of carnauba wax and polyethylene wax such as those marketed under the names “MicroCare 300®” and “310®” by the company Micro Powders, microwaxes consisting of a mixture of carnauba wax and synthetic wax such as those marketed under the name “MicroCare 325®” by the company Micro Powders, polyethylene microwaxes such as those marketed under the names “MicroPoly 200®”, “220®”, “220L®”, and “250S®”, by the company Micro Powders, and those marketed under the name “Cerapure H5-C” by the company Shamrock, or polypropylene microwaxes such as those marketed under the name “Mattewax” by the company Micro Powders; lauroyl lysine, starch, boron nitride, hollow polymer microspheres such as those of polyvinylidene chloride/acrylonitrile such as Expancel® (Nobel Industrie), acrylic acid copolymers, silicone resin powders, in particular silsesquioxane powders (silicone resin powders more particularly described in patent EP 293795; Tospearls® from Toshiba, for example), elastomeric polyorganosiloxane particles, precipitated calcium carbonate, magnesium carbonate and hydrocarbonate, hydroxyapatite, hollow silica microspheres, glass or ceramic microcapsules, metal soaps derived from organic carboxylic acids having from 8 to 22 carbon atoms, preferably from 12 to 18 carbon atoms, for example zinc, magnesium or lithium stearate, zinc laurate, magnesium myristate; barium sulphate and mixtures thereof.

According to one preferred embodiment the composition according to the invention may comprise a polytetrafluoroethylene (PTFE) powder.

The fillers may be present in the composition according to the invention in a total amount ranging from 0.1% to 30% by weight, relative to the total weight of the composition, preferably ranging from 0.5% to 20% by weight, and preferentially ranging from 0.8% to 10% by weight.

### Other Ingredients

The compositions may further comprise ingredients which are commonly used in cosmetology, such as vitamins, thickeners, trace elements, softeners, sequestrants, fragrances, alkalifying or acidifying agents, preservatives, sunscreens, antioxidants, fibres, care agents or mixtures of these.

The compositions according to the invention may comprise any cosmetic active agent, such as active agents chosen from antioxidants, preserving agents, fragrances, bactericidal or antiperspirant active agents, neutralizers, emollients, moisturizers, vitamins and screening agents, in particular sunscreens.

In one embodiment the first, second and, where appropriate, third compositions are lipstick compositions.

In another embodiment the first, second and, where appropriate, third compositions are eyelash or eyebrow coating compositions and more particularly mascaras.

In another embodiment the first, second and, where appropriate, third compositions are face or body skin coating compositions, more particularly face or body skin make-up compositions such as, for example, foundations or body make-up compositions.

A person skilled in the art may select the appropriate formulation, and also the method for preparing it, on the basis of his or her general knowledge, taking into account firstly the nature of the constituents used, especially their solubility in the vehicle, and secondly the intended use of each composition.

The invention is illustrated in greater detail by the examples described below. Unless otherwise mentioned, the amounts indicated are expressed as mass percentages.

In the composition examples described below the following two mixtures X′ and Y′, which are prepared by Dow Corning, are used:

MIXTURE X′:

| Ingredient (INCI name) | CAS No. | Amounts (%) | Function |
|---|---|---|---|
| Dimethyl Siloxane, | 68083-19-2 | 55-95 | polymer |
| Dimethylvinylsiloxy- | | | |
| terminated | | | |
| Silica Silylate | 68909-20-6 | 10-40 | filler |
| 1,3-Diethenyl-1,1,3,3- | 68478-92-2 | trace | catalyst |
| tetramethyldisiloxane | | | |
| complex | | | |
| Tetramethyldivinyl- | 2627-95-4 | 0.1-1 | polymer |
| disiloxane | | | |

MIXTURE Y′:

| Ingredient (INCI name) | CAS No. | Amounts (%) | Function |
|---|---|---|---|
| Dimethyl Siloxane, | 68083-19-2 | 55-95 | polymer |
| Dimethylvinylsiloxy | | | |
| Silica Silylate | 68909-20-6 | 10-40 | filler |
| Dimethyl, Methyl- | 68037-59-2 | 1-10 | polymer |
| hydrogeno Siloxane, | | | |
| trimethylsiloxy- | | | |
| terminated | | | |

The invention is illustrated in greater detail in the examples described below. Unless indicated otherwise, the amounts indicated are expressed as percentage by weight.

### EXAMPLE 1

### Lipsticks

Composition 1

| | % by weight |
|---|---|
| Isononyl isononanoate | 43.73 |
| Mixture X′ | 50 |
| Pigments | 6.27 |

Composition 2

| | % by weight |
|---|---|
| Isononyl isononanoate | 43.73 |
| Mixture Y′ | 50 |
| Pigments | 6.27 |

The first and second compositions above are mixed at the time of use in 50/50 proportion and then this mixture is applied to the lips. After a few minutes of drying, a comfortable film which does not transfer is obtained on the lips.

The transfer of the film obtained with the mixture of the 2 compositions is evaluated as described above. The film obtained from the mixture of compositions 1 and 2 has a transfer value of 0%.

The Persoz hardness of the film obtained with the mixture of the 2 compositions is evaluated as described above. The film obtained from the mixture of compositions 1 and 2 has a hardness value of 31, whereas the film obtained from the mixture of ingredients X′ and Y′ in 50/50 proportions is equal to 47.

### EXAMPLE 2

### Lipstick

Composition 1

| | % by weight |
|---|---|
| Polydimethylsiloxane* | 26.86 |
| Mixture X′ | 70 |
| Pigments | 3.14 |

Composition 2

| | % by weight |
|---|---|
| Polydimethylsiloxane* | 26.86 |
| Mixture Y′ | 70 |
| Pigments | 3.14 |

*PDMS: 5, 350, 50 000, 60 000 centiStokes

Compositions 1 and 2 were produced separately with the cSt, 350 cSt and 50 000 cSt and 60 000 cSt PDMS. They were mixed two by two at the time of use, as in accordance with Example 1; in other words, composition 1, with the 5 cSt PDMS, was mixed with composition 2, likewise comprising the 5 cSt PDMS, and likewise for the other PDMS used.

For each of the 4 mixtures prepared, after a few minutes of drying, a comfortable film which does not transfer is obtained on the lips.

The transfer of the film obtained was evaluated as described above for each of the 4 mixtures. The film obtained from the 4 mixtures of compositions 1 and 2, for each of the PDMS tested, has a transfer value of 0%.

### EXAMPLE 3

### Lipstick

Composition 1

| | % by weight |
|---|---|
| Hydrogenated isoparaffin | 16.87 |
| (6-8 moles of isobutylene) | |
| Mixture X′ | 80 |
| Pigments | 3.14 |

Composition 2

| | % by weight |
|---|---|
| Hydrogenated isoparaffin | 16.87 |
| (6-8 moles of isobutylene) | |
| Mixture Y′ | 80 |
| Pigments | 3.14 |

The first and second compositions above are mixed at the time of use in 50/50 proportion and then this mixture is applied to the lips. After a few minutes of drying, a comfortable film which does not transfer is obtained on the lips.

The transfer of the film obtained with the mixture of the 2 compositions is evaluated as described above. The film obtained from the mixture of compositions 1 and 2 has a transfer value of 0%.

### EXAMPLE 4

### Lipstick

Composition 1

| | % by weight |
|---|---|
| Polydecene | 16.87 |
| Mixture X′ | 80 |
| Pigments | 3.14 |

Composition 2

| | % by weight |
|---|---|
| Polydecene | 16.87 |
| Mixture Y′ | 80 |
| Pigments | 3.14 |

The first and second compositions above are mixed at the time of use in 50/50 proportion and then this mixture is applied to the lips. After a few minutes of drying, a comfortable film which does not transfer is obtained on the lips.

The transfer of the film obtained with the mixture of the 2 compositions is evaluated as described above. The film obtained from the mixture of compositions 1 and 2 has a transfer value of 0%.

### EXAMPLE 5

### Lipstick

Composition 1

| | % by weight |
|---|---|
| Isododecane | 18.25 |
| Mixture X′ | 50 |
| Phenyl trimethicone (20 cSt) | 23.61 |
| Trimethylsiloxyphenyl | 5 |
| dimethicone | |
| Pigments | 3.14 |

Composition 2

| | % by weight |
|---|---|
| Isododecane | 18.25 |
| Mixture Y′ | 50 |
| Phenyl trimethicone (20 cSt) | 23.61 |
| Trimethylsiloxyphenyl | 5 |
| dimethicone | |
| Pigments | 3.14 |

The first and second compositions above are mixed at the time of use in 50/50 proportion and then this mixture is applied to the lips. After a few minutes of drying, a comfortable film which does not transfer is obtained on the lips.

The transfer of the film obtained with the mixture of the 2 compositions is evaluated as described above. The film obtained from the mixture of compositions 1 and 2 has a transfer value of 0%.

### EXAMPLE 6

### Lipstick

Composition 1

| | % by weight |
|---|---|
| Cyclopentasiloxane | 18.25 |
| Mixture X′ | 50 |
| Phenyl trimethicone (20 cSt) | 23.61 |
| Trimethylsiloxyphenyl | 5 |
| dimethicone | |
| Pigments | 3.14 |

Composition 2

| | % by weight |
|---|---|
| Cyclopentasiloxane | 18.25 |
| Mixture Y′ | 50 |
| Phenyl trimethicone (20 cSt) | 23.61 |
| Trimethylsiloxyphenyl | 5 |
| dimethicone | |
| Pigments | 3.14 |

The first and second compositions above are mixed at the time of use in 50/50 proportion and then this mixture is applied to the lips. After a few minutes of drying, a comfortable film which does not transfer is obtained on the lips.

The transfer of the film obtained with the mixture of the 2 compositions is evaluated as described above. The film obtained from the mixture of compositions 1 and 2 has a transfer value of 0%.

### EXAMPLE 7

### Lipstick

Composition 1

| | % by weight |
|---|---|
| Hexafunctional polyester | 29 |
| acrylate (EBECRYL 870 from Cytec) | |
| Pt catalyst from Wacker | 50 |
| 5 cSt polydimethylsiloxane | 15.80 |
| Pigments | 5.2 |

Composition 2

| Ingredient (INCI name) | CAS No. | Amounts (%) |
|---|---|---|
| Dimethyl Siloxane, | 68083-19-2 | >60 |
| Dimethylvinylsiloxy | | |
| Dimethyl, | 68037-59-2 | 10-30 |
| Methylhydrogeno Siloxane, | | |
| trimethylsiloxy-terminated | | |

The first and second compositions above are mixed at the time of use in 60/40 proportion and then this mixture is applied to the lips. After a few minutes of drying, a comfortable film which does not transfer is obtained on the lips.

### Condensation Reaction Examples

MIXTURE X″:

| | | Content |
|---|---|---|
| Ingredient (INCI name) | CAS No | (%) |
| Bis-trimethoxysilylethyl tetra- | PMN871176 | 25-45 |
| methyldisiloxyethyl dimethicone | | |
| Disiloxane | 107-46-0 | 30-70 |
| Silica silylate | 68909-20-6 | 5-20 |

MIXTURE Y″:

| | | Content |
|---|---|---|
| Ingredient (INCI name) | CAS No | (%) |
| Tetra-t-butyl titanate | 3087-39-6 | 1-20 |
| Disiloxane | 107-46-0 | 80-99 |

### EXAMPLE 8

### Lipstick

Composition 1

| | % by weight |
|---|---|
| 5 cSt polydimethylsiloxane | 21.35 |
| Mixture X″ | 75.27 |
| Pigments | 3.38 |

Composition 2

| | % by weight |
|---|---|
| Mixture Y″ | 100 |

The first and second compositions above are mixed at the time of use in 93/7 proportion respectively and then this mixture is applied to the lips. After a few minutes of drying, a comfortable film which does not transfer is obtained on the lips.

The transfer of the film obtained with the mixture of the 2 compositions is evaluated as described above. The film obtained from the mixture of compositions 1 and 2 has a transfer value of 0%.

### EXAMPLE 9

### Lipstick

Composition 1

| | % by weight |
|---|---|
| Hydrogenated isoparaffin | 21.35 |
| (6-8 moles of isobutylene) | |
| Mixture X″ | 75.27 |
| Pigments | 3.38 |

Composition 2

| | % by weight |
|---|---|
| Mixture Y″ | 100 |

The first and second compositions above are mixed at the time of use in 93/7 proportion respectively and then this mixture is applied to the lips. After a few minutes of drying, a comfortable film which does not transfer is obtained on the lips.

The transfer of the film obtained with the mixture of the 2 compositions is evaluated as described above. The film obtained from the mixture of compositions 1 and 2 has a transfer value of 0%.

### EXAMPLE 10

### Lipstick

Composition 1

| | % by weight |
|---|---|
| Isononyl isonanoate | 21.35 |
| Mixture X″ | 75.27 |
| Pigments | 3.38 |

Composition 2

| | % by weight |
|---|---|
| Mixture Y″ | 100 |

The first and second compositions above are mixed at the time of use in 93/7 proportion and then this mixture is applied to the lips. After a few minutes of drying, a comfortable film which does not transfer is obtained on the lips.

The transfer of the film obtained with the mixture of the 2 compositions is evaluated as described above. The film obtained from the mixture of compositions 1 and 2 has a transfer value of 0%.

### EXAMPLE 11

### Lipstick

Composition 1

| | % by weight |
|---|---|
| Bis((3-methyldimethoxysilyl)propyl)- | 44 |
| polypropylene oxide (SIB 1660.0 from | |
| Gelest) | |
| Mixture X′ | 44 |
| Pigments | 3.5 |
| 5 cSt polydimethylsiloxane | 8.5 |

Composition 2

| | % by weight |
|---|---|
| Disiloxane (and) titanium catalyst | 100 |

The first and second compositions above are mixed at the time of use in 90/10 proportion and then this mixture is applied to the lips. After a few minutes of drying, a comfortable film which does not transfer is obtained on the lips.

The transfer of the film obtained with the mixture of the 2 compositions is evaluated as described above. The film obtained from the mixture of compositions 1 and 2 has a transfer value of 0%.

1. Make-up kit comprising at least two compositions which are packaged separately,
the kit comprising at least one compound X, at least one compound Y, at least one catalyst, at least one of the compounds, X or Y, being a silicone compound,
with the proviso that the compounds X and Y and the catalyst are not simultaneously in one of said compositions,
said compounds X and Y reacting together by a hydrosilylation reaction, when they are brought into contact with one another in the presence of the catalyst,
one at least of the first and second compositions comprising at least one compatible oil,
said compounds X and Y and said compatible oil, and their respective amounts, being such that the transfer index of a deposit comprising the two compositions is less than or equal to 40 out of 100.

2-41. (canceled)
